(19) United States
(12) Patent Application Publication
(10) Pub. No.: US 2020/0369667 A1
(43) Pub. Date: Nov. 26, 2020

(54) HETEROCYCLIC COMPOUNDS AS PRMT5 INHIBITORS

(71) Applicant: ANGEX PHARMACEUTICAL, INC., North Brunswick, NJ (US)

(72) Inventors: Wen-Lian Wu, Green Brook, NJ (US); Zhiqiang Yang, Westfield, NJ (US); Francis Lee, Yardley, PA (US); John Qiang Tan, North Brunswick, NJ (US)

(21) Appl. No.: 16/767,077

(22) PCT Filed: Nov. 1, 2018
(86) PCT No.: PCT/US2018/058721

§ 371 Date: May 26, 2020

Related U.S. Application Data
(60) Provisional application No. 62/594,898, filed on Dec. 5, 2017.

(51) Int. Cl. C07D 487/04; C07D 519/00; C07H 19/14

(52) U.S. Cl. CPC C07D 487/04; C07H 19/14; C07D 519/00

(57) ABSTRACT

The present disclosure describes novel PRMT5 inhibitors and methods for preparing them. The pharmaceutical compositions comprising such PRMTS inhibitors and methods of using them for treating cancer, infectious diseases, and other PRMTS associated disorders are also described.

### CROSS-REFERENCE TO RELATED APPLICATIONS

This application claims the benefit of U.S. Provisional Application No. 62/594,898, filed Dec. 5, 2017; which is incorporated by reference by its entirety.

### FIELD

The present disclosure relates to heterocyclic compounds, such as (1R,2S,3R,5S)-3-(4-amino-7H-pyrrolo[2,3-d]pyrimidine-7-yl)-5-(2-((S)-3-methyl-3,4-dihydro-2H-[1,4]oxazino[3,2-b]quinoline-7-yl)ethyl)cyclopentane-1,2-diol (1-8), as PRMT5 inhibitors, and pharmaceutical compositions comprising such compounds. The present disclosure also relates to the use of the compounds and compositions to treat cancer, infectious diseases and other disorders.

### BACKGROUND

Protein arginine N-methyltransferase 5 (PRMT5), the human homolog of Skb1 (Schizosaccharomyces pombe) and Hsl7 (Saccharomyces cerevisiae), was discovered in a yeast two-hybrid screen as a Janus kinase 2 (JAK2) binding protein. PRMT5 catalyzes the transfer of methyl group from the essential co-factor S-adenosylmethionine to methylate the arginine N-guanidine group of various proteins. Substrate proteins for PRMT5 include histones, transcriptional elongation factors, kinases, and tumor suppressors, for example, histone H4, histone H3, and non-histone proteins such as FGF-216, NF-kB17, HOXA918, and p53. PRMT5 is involved in the transcriptional repression of a number of tumor suppressor genes including suppressor of tumorigenicity 7 (ST7), nonmetastatic 23 (NM23), retinoblastoma (Rb) family, and programmed cell death 4 (PDCD4).

PRMT5 has recently emerged as a promising drug target due to its frequent overexpression in a variety of malignancies including glioma, lung cancer, melanoma, mantle cell lymphoma, multiple endocrine neoplasia, prostate and gastric cancer, as well as its synthetic lethal relationship with methylthioadenosine phosphorylase (MTAP). Importantly, in addition to overexpression, PRMT5 localization differs between normal and tumor tissues and between tumor subtypes. This is indicative that its compartment-specific functions likely regulate distinct molecular programs and are therefore associated with diverse phenotypic outcomes. Thus, the identification and development of small-molecules that inhibit PRMT5 activity will serve as therapeutic approach for the treatment of a variety of PRMT5-related diseases or disorders, such as cancer.

### SUMMARY

This disclosure relates to heterocyclic compounds comprising at least three ring systems, such as certain optionally substituted 7-(2-((1S,2R,3S,4R)-2,3-dihydroxy-4-(7H-pyrrolo[2,3-d]pyrimidin-7-yl)cyclopentyl)ethyl)quinazolin-4(3H)-one, optionally substituted (1S,2R,3S,5R)-3-(2-(3,4-dihydro-2H-[1,4]oxazino[3,2-b]quinolin-7-yl)ethyl)-5-(7H-pyrrolo[2,3-d]pyrimidin-7-yl)cyclopentane-1,2-diol, optionally substituted 7-(2-((1S,2R,3S,4R)-2,3-dihydroxy-4-(7H-pyrrolo[2,3-d]pyrimidin-7-yl)cyclopentyl)ethyl)-2H-[1,4]oxazino[3,2-b]quinolin-3(4H)-one, optionally substituted (1S,2R,3S,5R)-3-(2-(3,4-dihydro-2H-[1,4]thiazino[3,2-b]quinolin-7-yl)ethyl)-5-(7H-pyrrolo[2,3-d]pyrimidin-7-yl)cyclopentane-1,2-diol, optionally substituted 6-(2-((1S,2R,3S,4R)-2,3-dihydroxy-4-(7H-pyrrolo[2,3-d]pyrimidin-7-yl)cyclopentyl)ethyl)-2H-benzo[e][1,2,4]thiadiazine 1,1-dioxide, optionally substituted (1R,2S,3R,5S)-3-(7H-pyrrolo[2,3-d]pyrimidin-7-yl)-5-(3-(quinolin-7-yl)propyl)cyclopentane-1,2-diol, optionally substituted 7-(2-((1S,2R,3S,4R)-2,3-dihydroxy-4-(7H-pyrrolo[2,3-d]pyrimidin-7-yl)cyclopentyl)ethyl)-2H-[1,4]thiazino[3,2-b]quinolin-3(4H)-one, optionally substituted 8-(2-((1S,2R,3S,4R)-2,3-dihydroxy-4-(7H-pyrrolo[2,3-d]pyrimidin-7-yl)cyclopentyl)ethyl)-2,3-dihydroimidazo[2,1 -b]quinazolin-5(1 H)-one, optionally substituted (2R,3S,4R,5R)-2-(2-(2H-benzo[b][1,4]oxazin-6-yl)ethyl)-5-(7H-pyrrolo[2,3-d]pyrimidin-7-yl)tetrahydrofuran-3,4-diol, optionally substituted 6-(2-((1S,2R,3S,4R)-2,3-dihydroxy-4-(7H-pyrrolo[2,3-d]pyrimidin-7-yl)cyclopentyl)ethyl)-2H-benzo[b][1,4]thiazine 1,1-dioxide, or any compound described herein.

Some embodiments include a compound represented by Formula 1:

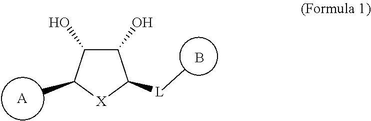

or a pharmaceutically acceptable salt thereof; wherein

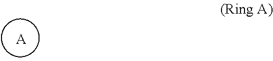

is an optionally substituted 9-membered bicyclic aromatic heterocyclic ring system containing 1, 2, 3, 4, 5, or 6 ring nitrogen atoms;

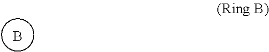

is an optionally substituted fused bicyclic or tricyclic heterocyclic ring system containing 1, 2, 3, 4, 5, or 6 ring heteroatoms independently selected from N, O and S; X is —O—, —CH₂-, or —CF₂-; L is optionally substituted C₁₋₃ hydrocarbylene, optionally substituted —O—C₁₋₂ hydrocarbylene-, optionally substituted —S—C₁₋₂ hydrocarbylene-, or optionally substituted —NR^{A}—C₁₋₂ hydrocarbylene; and R^{A} is H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl.

Some embodiments include use of a compound described herein, or a pharmaceutically acceptable salt thereof (referred to collectively herein as a “subject compound”) in the manufacture of a medicament for the treatment of cancer, infectious diseases, and other PRMTS related disorders.

Some embodiments include a pharmaceutical composition comprising a therapeutically effective amount of a subject compound in combination with at least one pharmaceutically acceptable carrier.

Some embodiments include a process for making a pharmaceutical composition comprising combining a subject compound and at least one pharmaceutically acceptable carrier.

Some embodiments include a method of treating cancer, infectious diseases, and other PRMTS related disorders comprising administering a subject compound to a patient in need thereof.

Some embodiments include use of a subject compound in the manufacture of a medicament for the treatment of cancer, infectious diseases, and other PRMTS related disorders.

### DETAILED DESCRIPTION

Unless otherwise indicated, any reference to a compound herein by structure, name, or any other means, includes pharmaceutically acceptable salts, such as sodium, potassium, and ammonium salts; prodrugs, such as ester prodrugs; alternate solid forms, such as polymorphs, solvates, hydrates, etc.; tautomers; or any other chemical species that may rapidly convert to a compound described herein under conditions in which the compounds are used as described herein.

If stereochemistry is not indicated, a name or structural depiction includes any stereoisomer or any mixture of stereoisomers.

In some embodiments, a compound of Formula 1 is a single enantiomer.

Unless otherwise indicated, when a compound or chemical structural feature such as aryl is referred to as being “optionally substituted”, it includes a feature that has no substituents (i.e. unsubstituted), or a feature that is “substituted”, meaning that the feature has one or more substituents. The term “substituent” is broad, and includes a moiety that occupies a position normally occupied by one or more hydrogen atoms attached to a parent compound or structural feature. In some embodiments, a substituent may be an ordinary organic moiety known in the art, which may have a molecular weight (e.g. the sum of the atomic masses of the atoms of the substituent) of 15 g/mol to 50 g/mol, 15 g/mol to 60 g/mol, 15 g/mol to 70 g/mol, 15 g/mol to 80 g/mol, 15 g/mol to 90 g/mol, 50 g/mol to 60 g/mol, 60 g/mol to 70 g/mol, 70 g/mol to 80 g/mol, 80 g/mol to 90 g/mol, 90 g/mol to 100 g/mol, 15 g/mol to 100 g/mol, 15 g/mol to 150 g/mol, 15 g/mol to 200 g/mol, 15 g/mol to 300 g/mol, or 15 g/mol to 500 g/mol. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, P, Si, F, Cl, Br, or I; provided that the substituent includes at least one C, N, O, S, P, Si, F, Cl, Br, or I atom and N, S and P can be optionally oxidized. Examples of substituents include, but are not limited to, deuterium, tritium, alkyl, alkenyl, alkynyl, heteroalkyl, heteroalkenyl, heteroalkynyl, aryl, heteroaryl, hydroxy, alkoxy, aryloxy, acyl, acyloxy, alkylcarboxylate, thiol, alkylthio, cyano, halo, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, nitro, N-oxide, silyl, sulfenyl, sulfinyl, sulfonyl, sulfoxide, haloalkyl, haloalkoxyl, trihalomethanesulfonyl, trihalomethanesulfonamido, amino, phosphonic acid, etc.

For convenience, the term “molecular weight” is used with respect to a moiety or part of a molecule to indicate the sum of the atomic masses of the atoms in the moiety or part of a molecule, even though it may not be a complete molecule.

The structures associated with some of the chemical names referred to herein are depicted below. These structures may be unsubstituted, as shown below, or substituted with a substituent that may independently be in any position normally occupied by a hydrogen atom when the structure is unsubstituted. Unless a point of attachment is indicated by

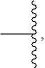

attachment may occur at any position normally occupied by a hydrogen atom.

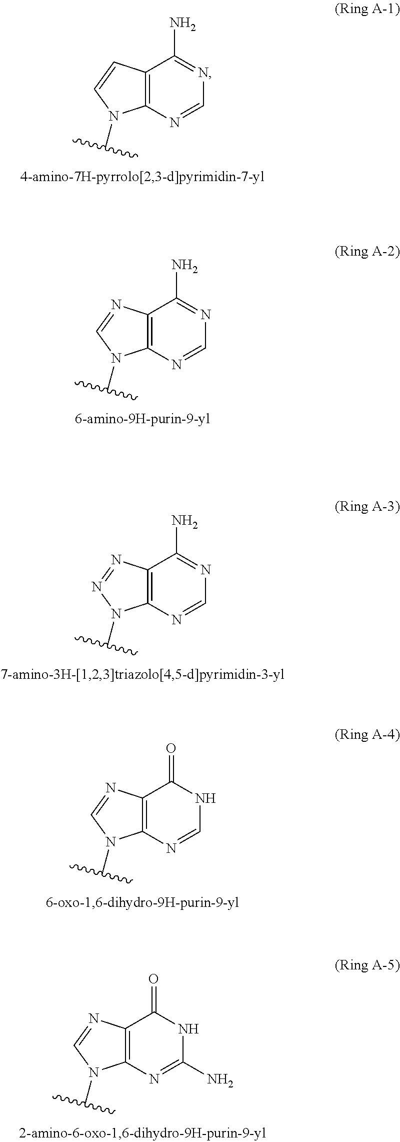 [SMILES: *n1ccc2c(N)ncnc21 ; *n1cnc2c(N)ncnc21 ; *n1nnc2c(N)ncnc21 ; *n1cnc2c(=O)[nH]cnc21 ; *n1cnc2c(=O)[nH]c(N)nc21]

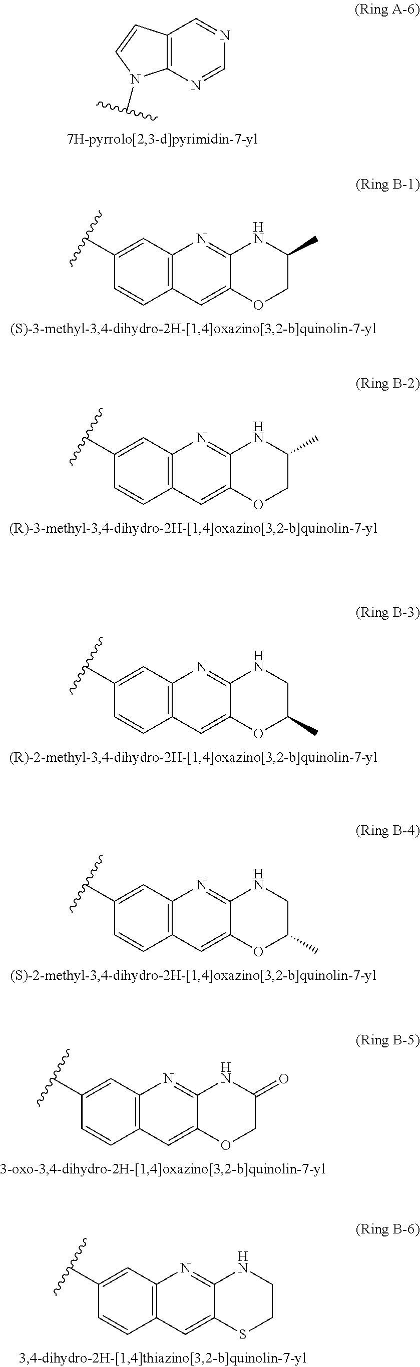

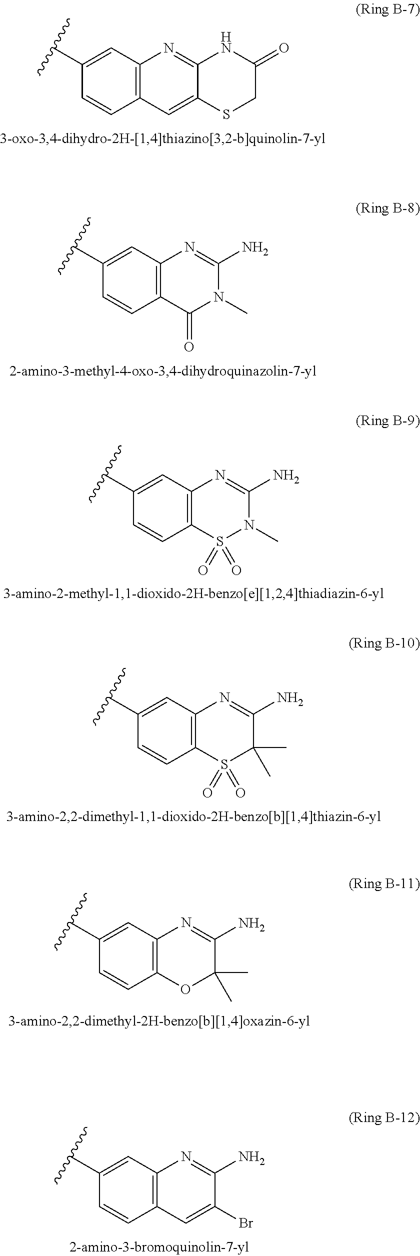 [SMILES: *c1ccc2cc3c(nc2c1)NC(=O)CS3 ; *c1ccc2c(=O)n(C)c(N)nc2c1 ; *c1ccc2c(c1)N=C(N)N(C)S2(=O)=O ; *c1ccc2c(c1)N=C(N)C(C)(C)S2(=O)=O ; *c1ccc2c(c1)N=C(N)C(C)(C)O2 ; *c1ccc2cc(Br)c(N)nc2c1]

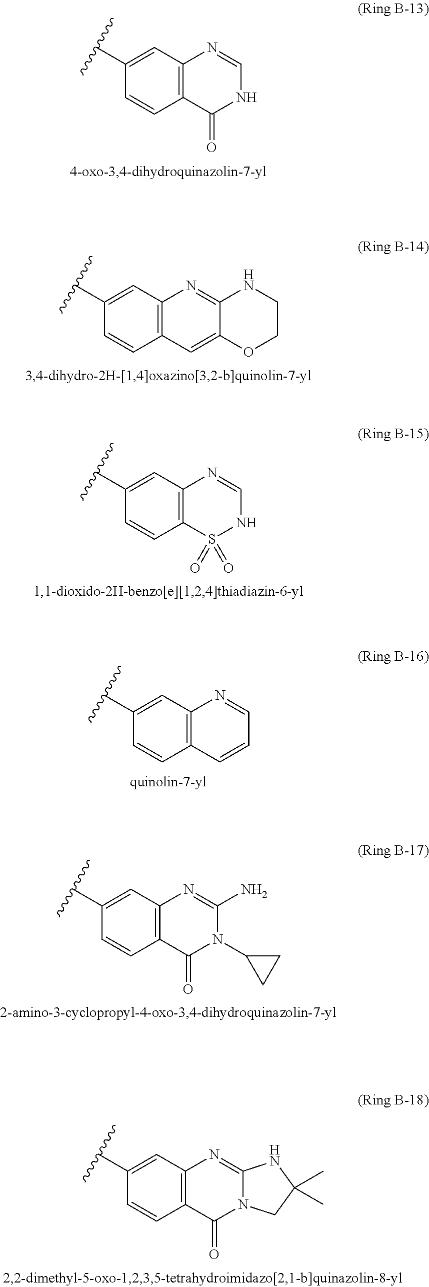 [SMILES: *c1ccc2c(=O)[nH]cnc2c1 ; *c1ccc2cc3c(nc2c1)NCCO3 ; *c1ccc2c(c1)N=CNS2(=O)=O ; *c1ccc2cccnc2c1 ; *c1ccc2c(=O)n(C3CC3)c(N)nc2c1 ; *c1ccc2c(=O)n3c(nc2c1)NC(C)(C)C3]

In some embodiments, Ring A of Formula 1 comprises:

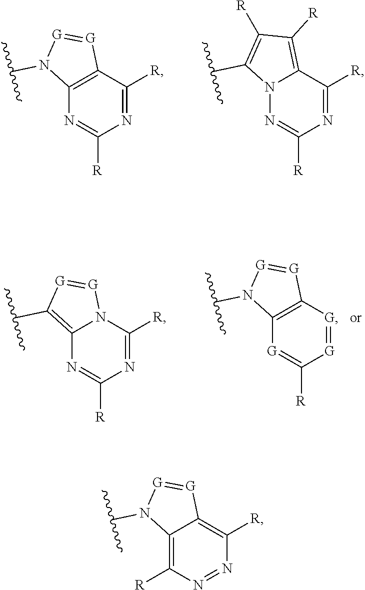

and Ring B comprises:

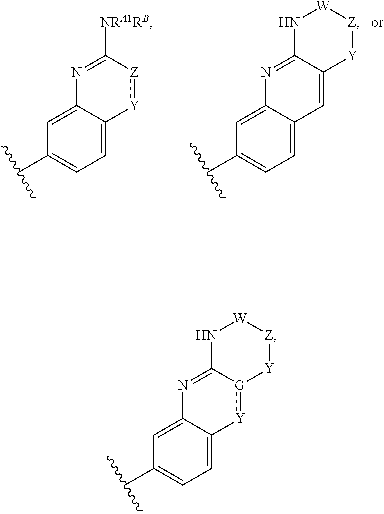

wherein each structure is optionally substituted; each G is independently N or CR; the dashed line represents optionally with or without a bond. Each Y is independently a bond, —C(R^{C}R^{D})—, —C (═O)—, —O—, —N(R^{A})-, or —S(O)₀₋₂-; Z is —C(R^{C}R^{D})—, —C(═O)—, —O—, —N(R^{A})-, or —S(O)₀₋₂-; W is —C(R^{C}R^{D})—, —O (═O)-, or —SO₂-; each R is independently H, F, Cl, Br, I, —NR^{A}R^{B}, C₁₋₆ hydrocarbyl, —OH, —CN, or —O—C₁-6 alkyl; each R^{C}, and each R^{D} is independently H, F, Cl, Br, I, —NR^{A}R^{B}, C₁₋₆ hydrocarbyl, —OH, —CN, or —O—C₁₋₆ alkyl; each R^{A} and R^{A1} are independently H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆alkyl; R^{B} is H, C₁₋₆hydrocarbyl, C₁₋₆ heteroaryl, C₁₋₆ heterocycloalkyl, —C(O)—C₁₋₆ alkyl, —C(O)NH—C₁₋₆ alkyl, or —C (O)OC₁₋₆ alkyl; and R^{A1} and Z or substituent of Z may connect and together with the ring containing Z to form a fused ring.

With respect to any relevant structural representation, such as Formula 1, Ring A is an optionally substituted 9-membered bicyclic aromatic heterocyclic ring system containing 1, 2, 3, 4, 5, or 6 ring nitrogen atoms, such as an optionally substituted 5-membered heteroaryl ring having 1, 2, or 3 ring nitrogen atoms that is fused with an optionally substituted 6-membered aromatic ring including optionally substituted 6-membered aromatic all carbon ring or optionally substituted 6-membered heteroaryl ring having 1, 2, or 3 ring nitrogen atoms. In some embodiments, any or each of the substituents of Ring A may have a molecular weight of 15 g/mol to 50 g/mol, 60 g/mol, 70 g/mol, 80 g/mol, 90 g/mol, 100 g/mol, or 300 g/mol. Potential substituents of Ring A may include —OH; —CN; halo, such as F, Cl, Br, I; hydrocarbyl, such as methyl, C₂ alkyl, C₂ alkenyl, C₂ alkynyl, C₃ alkyl, C₃ cycloalkyl, C₃ alkenyl, C₃ alkynyl, C₄ alkyl, C₄ cycloalkyl, C₄ alkenyl, C₄ alkynyl, C₅ alkyl, C₅ cycloalkyl, C₅ alkenyl, C₅ alkynyl, C₆ alkyl, C₆ cycloalkyl, C₆ alkenyl, C₆ alkynyl, phenyl, etc.; CN₀₋₁O₀₋₂F₀₋₃H₀₋₄; C₂N₀₋₁O₀₋₃F₀₋₅H₀₋₆; C₃N₀₋₁O₀₋₃F₀₋₇H₀₋₈;l C₄N₀₋₁O₀₋₃F₀₋₉H_{0-10;} C₅N₀₋₁O₀₋₃F₀₋₁₁H_{0-12;} C₆N₀₋₁O₀₋₃F₀₋₁₃H_{0-14;} etc. In some embodiments, Ring A has a substituent of NH2 at position 4 of Formula A as shown below. In some embodiments, Ring A is optionally substituted 7H-pyrrolo[2,3-d]pyrimidin-7-yl having 1, 2, 3, or 4 substituents, such as 7H-pyrrolo[2,3-d]pyrimidin-7-yl substituted with F, Cl, Br, C₁₋₆ alkyl, —CO₂H, —CN, —CO—C₁₋₆-alkyl, —C(O)O—C₁₋₆-alkyl, C₁₋₆ alkyl-OH, OH, NH₂, etc. In some embodiments, Ring A is optionally substituted 4-amino-7H-pyrrolo[2,3-d]pyrimidin-7-yl. In some embodiments, Ring A is unsubstituted 4-amino-7H-pyrrolo[2,3-d]pyrimidin-7-yl.

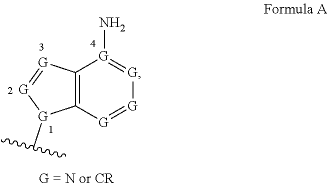

With respect to Formula 1, in some embodiments, Ring A is represented by Formula A1, A2, A3, A4, or A5:

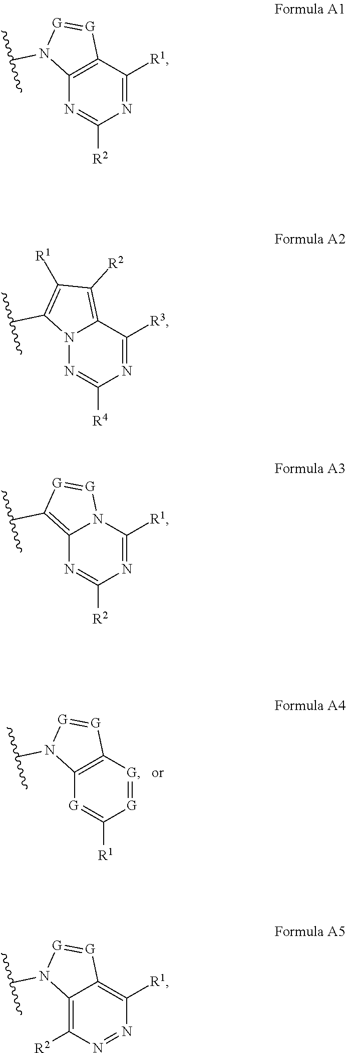

With respect to any relevant structural representation, such as Formula A1, A2, A3, A4 or A5, R¹ is H or any substituent, such as R^{A}, F, Cl, —CN, ═O, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. Some of the structures with attachment points are shown below. In some embodiments, R¹ may be H; F; Cl; —CN; CF₃; OH; NH₂; C₁₋₆ alkyl, such as methyl, ethyl, any one of the propyl isomers (e.g. n-propyl and isopropyl), cyclopropyl, any one of the butyl isomers, any one of the cyclobutyl isomers (e.g. cyclobutyl and methylcyclopropyl), any one of the pentyl isomers, any one of the cyclopentyl isomers, any one of the hexyl isomers, and any one of the cyclohexyl isomers, etc.; or C₁₋₆ alkoxy, such as —O-methyl, —O-ethyl, any one of the isomers of —O-propyl, —O-cyclopropyl, any one of the isomers of —O-butyl, any one of the isomers of —O-cyclobutyl, any one of the isomers of —O-pentyl, any one of the isomers of —O-cyclopentyl, any one of the isomers of —O-hexyl, any one of the isomers of —O-cyclohexyl, etc. In some embodiments, R¹ may be H, F, CI, or NH₂. In some embodiments, R¹ may be H. In some embodiments, R¹ is NH₂.

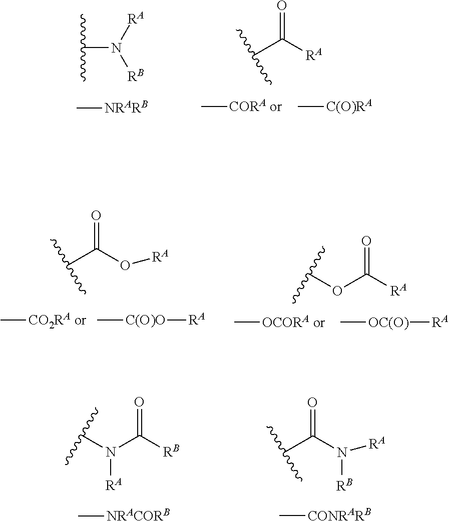

With respect to any relevant structural representation, each R^{A} may independently be H, or C₁₋₁₂ hydrocarbyl, such as C₁₋₁₂ alkyl, C₁₋₁₂ alkenyl, C₁₋₁₂ alkynyl, phenyl, etc., including: linear or branched alkyl having a formula CₐH₂ₐ₊₁, or cycloalkyl having a formula CₐH₂ₐ₋₁, wherein a is 1, 2, 3, 4, 5, 6, 7, 8, 9, 10, 11, or 12, such as linear or branched alkyl with a formula: CH₃, C₂H₅, C₃H₇, C₄H₉, C₅H₁₁, C₆H₁₃, C₇H₁₅, C₈H₁₇, C₉H₁₉, C₁₀H_{21,} etc., or cycloalkyl with a formula: C₃H₅, C₄H₇, C₅H₉, C₆H₁₁, C₇H₁₃, C₅H₁₅, C₉H₁₇, C₁₀H₁₉, etc. In some embodiments, R^{A} may be H or C₁₋₆ alkyl. In some embodiments, R^{A} may be H or C₁₋₃ alkyl. In some embodiments, R^{A} may be H or CH₃. In some embodiments, R^{A} may be H.

With respect to any relevant structural representation, each R^{A1} may independently be H, or C₁₋₁₂ hydrocarbyl, such as C₁₋₁₂ alkyl, C₁₋₁₂ alkenyl, C₁₋₁₂ alkynyl, phenyl, etc., including: linear or branched alkyl having a formula CₐH₂ₐ₊₁, or cycloalkyl having a formula CₐH₂ₐ₋₁, wherein a is 1, 2, 3, 4, 5, 6, 7, 8, 9, 10, 11, or 12, such as linear or branched alkyl with a formula: CH₃, C₂H₅, C₃H₇, C₄H₉, C₅H₁₁, C₆H₁₃, C₇H₁₅, C₅H₁₇, C₉H₁₉, C₁₀H₂₁, etc., or cycloalkyl with a formula: C₃H₅, C₄H₇, C₅H₉, C₆H₁₁, C₇H₁₃, C₅H₁₅, C₉H₁₇, C₁₀H₁₉, etc. In some embodiments, R^{A1} may be H or C₁₋₆ alkyl. In some embodiments, R^{A1} may be H or C₁₋₃ alkyl. In some embodiments, R^{A1} may be H or CH₃. In some embodiments, R^{A1} may be H.

With respect to any relevant structural representation, each R^{B} may independently be H, or C₁₋₁₂ hydrocarbyl, such as C₁₋₁₂ alkyl, C₁₋₁₂ alkenyl, C₁₋₁₂ alkynyl, phenyl, etc., including: linear or branched alkyl having a formula CₐH₂ₐ₊₁, or cycloalkyl having a formula CₐH₂ₐ₋₁, wherein a is 1, 2, 3, 4, 5, 6, 7, 8, 9, 10, 11, or 12, such as linear or branched alkyl with a formula: CH₃, C₂H₅, C₃H₇, C₄H₉, C₅H₁₁, C₆H₁₃, C₇H₁₅, C₉H₁₇, C₉H₁₉, C₁₀H₂₁, etc., or cycloalkyl with a formula: C₃H₅, C₄H₇, C₅H₉, C₆H₁₁, C₇H₁₃, C₉H₁₅, C₉H₁₇, C₁₀H₁₉, etc. In some embodiments, R^{B} may be H or C₁₋₃ alkyl. In some embodiments, R^{B} may be H or CH₃. In some embodiments, R^{B} may be H.

With respect to any relevant structural representation, such as Formula A1, A2, A3, or A5, R² is H or any substituent, such as R^{A}, F, Cl, —CN, ═O, —ORA, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R² may be H, F, Cl, CN, CF₃, OH, NH₂, C₁₋₆ alkyl, or C₁₋₆ alkoxy. In some embodiments, R² may be H, F, Cl, or NH₂. In some embodiments, R² may be H. In some embodiments, R² may be NH₂.

With respect to any relevant structural representation, such as Formula A2, R³ is H or any substituent, such as R^{A}, F, Cl, —CN, ═O, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R³ may be H, F, Cl, —CN, CF₃, OH, NH₂, C₁₋₆ alkyl, or C₁₋₆ alkoxy. In some embodiments, R³ may be H, F, Cl, or NH₂. In some embodiments, R³ may be H. In some embodiments, R³ may be NH₂.

With respect to any relevant structural representation, such as Formula A1, A3, A4, or A5, G is independently N or CR, wherein R is H or any substituent, such as R^{A}, F, Cl, —CN, ═O, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, G is N. In some embodiments, G is CR. In some embodiments, R may be H, F, Cl, —CN, CF₃, OH, NH₂, C₁₋₆ alkyl, or C₁₋₆ alkoxy. In some embodiments, R may be H, F, Cl, or NH₂. In some embodiments, R may be H. In some embodiments, R may be NH₂.

With respect to any relevant structural representation, such as Formula A1, in some embodiments, each G is CR and R¹ is NH₂. In some embodiments, each R and R² are all H. In some embodiments, R¹ is NH₂, R² is H, and each R is H.

With respect to any relevant structural representation, such as Formula 1, Ring B is an optionally substituted fused bicyclic heterocyclic ring system or fused tricyclic heterocyclic ring system containing 1, 2, 3, 4, 5, or 6 ring heteroatoms independently selected from N, O and S. In some embodiments, Ring B is optionally substituted fused bicyclic heterocyclic ring system. In some embodiments, Ring B is fused tricyclic heterocyclic ring system. In some embodiments, any or each of the substituents of Ring B may have a molecular weight of 15 g/mol to 50 g/mol, 50 g/mol to 100 g/mol, 50 g/mol to 75 g/mol, 75 g/mol to 100 g/mol, or 100 g/mol to 300 g/mol. Potential substituents of Ring B may include halo, such as F, Cl, Br, or I; hydrocarbyl, such as methyl, C₂ alkyl, C₂ alkenyl, C₂ alkynyl, C₃ alkyl, C₃ cycloalkyl, C₃ alkenyl, C₃ alkynyl, C₄ alkyl, C₄ cycloalkyl, C₄ alkenyl, C₄ alkynyl, C₅ alkyl, C₅ cycloalkyl, C₅ alkenyl, C₅ alkynyl, C₆ alkyl, C₆ cycloalkyl, C₆ alkenyl, C₆ alkynyl, or phenyl, etc.; CN₀₋₁O₀₋₂F₀₋₃H₀₋₄; C₂N₀₋₁O₀₋₃F₀₋₅H₀₋₆; C₃N₀₋₁O₀₋₃F₀₋₇H₀₋₈; C₄N₀₋₁O₀₋₃F₀₋₉H_{0-10;} C₅N₀₋₁O₀₋₃F₀₋₁₁ H_{0-12;} or C₆N₀₋₁O₀₋₃F₀₋₁₃H₀₋₁₄; etc. In some embodiments, Ring B is optionally substituted 4-dihydro-2H-[1,4]oxazino[3,2-b]quinolin-7-yl, optionally substituted 3-oxo-3,4-dihydro-2H-[1,4]oxazino[3,2-b]quinolin-7-yl, 3,4-dihydro-2H-[1,4]thiazino[3,2-b]quinolin-7-yl, optionally substituted 3-oxo-3,4-dihydro-2H-[1,4]thiazino[3,2-b]quinolin-7-yl, optionally substituted 3-oxo-1,2,3,4-tetrahydropyrazino[2,3-b]quinolin-7-yl, optionally substituted 3-oxo-3,4-dihydro-2H-[1,4]oxazino[3,2-b]quinolin-7-yl, optionally substituted 2-oxo-1,2,3,4-tetrahydropyrimido[4,5-b]quinolin-8-yl, optionally substituted 2-oxo-1,4-dihydro-2H-[1,3]thiazino[4,5-b]quinolin-8-yl, optionally substituted 2-oxo-1,4-dihydro-2H-[1,3]oxazino[4,5-b]quinolin-8-yl, optionally substituted 2,4-dioxo-1,2,3,4-tetrahydropyrimido[4,5-b]quinolin-8-yl, optionally substituted 1,1-dioxido-3-oxo-3,4-dihydro-2H-[1,2,4]thiadiazino[5,6-b]quinolin-7-yl, optionally substituted 2,2-dioxido-3,4-dihydro-1H-[1,2]thiazino[3,4-b]quinolin-8-yl, optionally substituted 2,6-dioxo-1,3,4,6-tetrahydro-2H-pyrimido[2,1-b]quinazolin-9-yl, optionally substituted 2-oxo-2,3-dihydro-1H-imidazo[4,5-b]quinolin-6-yl, optionally substituted 2-oxo-2,3-dihydrothiazolo[4,5-b]quinolin-6-yl, optionally substituted 2-oxo-2,3-dihydro-1 H-pyrrolo[2,3-b]quinolin-7-yl, optionally substituted 4-oxo-3,4-dihydroquinazolin-7-yl, optionally substituted 1,1-dioxido-2H-benzo[e][1,2,4]thiadiazin-6-yl, optionally substituted 1,1-dioxido-2H-benzo[b][1,4]thiazin-6-yl, optionally substituted 2H-benzo[b][1,4]oxazin-6-yl, optionally substituted 3H-indol-6-yl, or optionally substituted quinolin-7-yl. In some embodiments, Ring B is optionally substituted 3,4-dihydro-2H-[1,4]oxazino[3,2-b]quinolin-7-yl having 0, 1, 2, or 3, 4, 5, 6, 7, 8, 9 substituents, such as 3,4-dihydro-2H-[1,4]oxazino[3,2-b]quinolin-7-ylsubstituted with F, Cl, Br, C₁₋₆ alkyl, —CO₂H, —CN, —CO—C₁₋₆-alkyl, —C(O)O—C₁₋₆-alkyl, —C₁₋₆alkyl-OH, OH, NH₂, etc. In some embodiments, Ring B is 3,4-dihydro-2H-[1,4]oxazino[3,2-b]quinolin-7-yl having 2 substituents. In some embodiments, Ring B is 3,4-dihydro-2H-[1,4]oxazino[3,2-b]quinolin-7-yl having 1 substituent. In some embodiments, Ring B is unsubstituted 3,4-dihydro-2H-[1,4]oxazino[3,2-b]quinolin-7-yl. In some embodiments, Ring B is 3,4-dihydro-2H-[1,4]oxazino[3,2-b]quinolin-7-yl having 1 substituent that is methyl.

In some embodiments, Ring B is (S)-3-methyl-3,4-dihydro-2H-[1,4]oxazino[3,2-b]quinolin-7-yl. In some embodiments, Ring B is (R)-3-methyl-3,4-dihydro-2H-[1,4]oxazino[3,2-b]quinolin-7-yl. In some embodiments, Ring B is (R)-2-methyl-3,4-dihydro-2H-[1,4]oxazino[3,2-b]quinolin-7-yl. In some embodiments, Ring B is (S)-2-methyl-3,4-dihydro-2H-[1,4]oxazino[3,2-b]quinolin-7-yl. In some embodiments, Ring B is 3-oxo-3,4-dihydro-2H-[1,4]oxazino[3,2-b]quinolin-7-yl. In some embodiments, Ring B is 3,4-dihydro-2H-[1,4]thiazino[3,2-b]quinolin-7-yl. In some embodiments, Ring B is 3-oxo-3,4-dihydro-2H-[1,4]thiazino[3,2-b]quinolin-7-yl. In some embodiments, Ring B is 2,2-dimethyl-3-oxo-1,2,3,4-tetrahydropyrazino[2,3-b]quinolin-7-yl. In some embodiments, Ring B is 2,2-dimethyl-3-oxo-3,4-dihydro-2H-[1,4]oxazino[3,2-b]quinolin-7-yl. In some embodiments, Ring B is 4,4-dimethyl-2-oxo-1,4-dihydro-2H-[1,3]thiazino[4,5-b]quinolin-8-yl. In some embodiments, Ring B is 3-methyl-2,4-dioxo-1,2,3,4-tetrahydropyrimido[4,5-b]quinolin-8-yl. In some embodiments, Ring B is 2-methyl-1,1-dioxido-3-oxo-3,4-dihydro-2H-[1,2,4]thiadiazino[5,6-b]quinolin-7-yl. In some embodiments, Ring B is 2,2-dioxido-3,4-dihydro-1 H-[1,2]thiazino[3,4-b]quinolin-8-yl. In some embodiments, Ring B is 2,6-dioxo-1,3,4,6-tetrahydro-2H-pyrimido[2,1-b]quinazolin-9-yl. In some embodiments, Ring B is 2-oxo-2,3-dihydro-1 H-imidazo[4,5-b]quinolin-6-yl. In some embodiments, Ring B is 2-oxo-2,3-dihydrothiazolo[4,5-b]quinolin-6-yl. In some embodiments, Ring B is 3,3-dimethyl-2-oxo-2,3-dihydro-1H-pyrrolo[2,3-b]quinolin-7-yl. In some embodiments, Ring B is 2-amino-3-methyl-4-oxo-3,4-dihydroquinazolin-7-yl. In some embodiments, Ring B is 3-amino-2-methyl-1,1-dioxido-2H-benzo[e][1,2,4]thiadiazin-6-yl. In some embodiments, Ring B is 3-amino-2,2-dimethyl-1,1-dioxido-2H-benzo[b][1,4]thiazin-6-yl. In some embodiments, Ring B is 3-amino-2,2-dimethyl-2H-benzo[b][1,4]oxazin-6-yl. In some embodiments, Ring B is 2-amino-3,3-dimethyl-3H-indol-6-yl. In some embodiments, Ring B is 2-amino-3-bromoquinolin-7-yl. In some embodiments, Ring B is 2-amino-3-cyclopropyl-4-oxo-3,4-dihydroquinazolin-7-yl. In some embodiments, Ring B is 2,2-dimethyl-5-oxo-1,2,3,5-tetrahydroimidazo[2,1-b]quinazolin-8-yl.

In some embodiments, Ring B is represented by formula 2, 3, or 4:

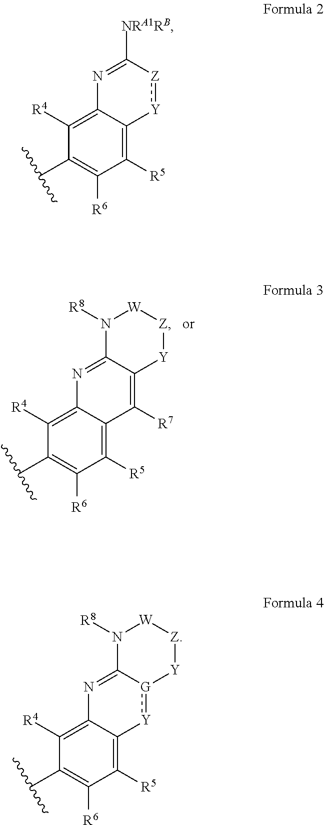

With respect to any relevant structural representation, such as Formula 2, the dashed line represents optionally with or without a bond. In some embodiments, Y and Z is linked by a single bond. In some embodiments, Y and Z is linked by a double bond. In some embodiments, YZ is —CH═CH-. In some embodiments, YZ is —CH═C(Br)-. In some embodiments, YZ is —CH═C(Br)-, wherein Y is CH, and Z is CBr.

With respect to any relevant structural representation, such as Formula 4, the dashed line represents optionally with or without a bond. In some embodiments, Y and G is linked by a single bond. In some embodiments, Y and G is linked by a double bond. In some embodiments, YG is —CH═CH-. In some embodiments, YG is C(O)—N.

With respect to any relevant structural representation, such as Formula 2, 3, or 4, R⁴ is H or any substituent, such as R^{A}, F, Cl, CN, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R⁴ may be H, F, Cl, CN, CF₃, OH, NH₂, C₁₋₆ alkyl, or C₁₋₆ alkoxy. In some embodiments, R⁴ may be H, F, or Cl. In some embodiments, R⁴ may be H.

With respect to any relevant structural representation, such as Formula 2, 3, or 4, R⁵ is H or any substituent, such as RA, F, Cl, CN, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R⁵ may be H, F, Cl, CN, CF₃, OH, NH₂, C₁₋₆ alkyl, or C₁₋₆ alkoxy. In some embodiments, R⁵ may be H, F, or Cl. In some embodiments, R⁵ may be H.

With respect to any relevant structural representation, such as Formula 2, 3, or 4, R⁶ is H or any substituent, such as RA, F, Cl, CN, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R⁶ may be H, F, Cl, CN, CF₃, OH, NH₂, C₁₋₆ alkyl, or C₁₋₆ alkoxy. In some embodiments, R⁶ may be H, F, or Cl. In some embodiments, R⁶ may be H.

With respect to any relevant structural representation, such as Formula 3, R⁷ is H or any substituent, such as R^{A}, F, Cl, CN, —OR^{A}, CF₃, —NO₂, —NR^{A}R^{B}, —COR^{A}, —CO₂R^{A}, —OCOR^{A}, —NR^{A}COR^{B}, or —CONR^{A}R^{B}, etc. In some embodiments, R⁷ may be H, F, Cl, CN, CF₃, OH, NH₂, C₁₋₆ alkyl, or C₁₋₆ alkoxy. In some embodiments, R⁷ may be H, F, or Cl. In some embodiments, R⁷ may be H.

With respect to any relevant structural representation, such as Formula 3 or 4, R⁸ is H or any substituent, such as R^{A}, OH, CF₃, —COR^{A}, —CO₂R^{A}, or —CONR^{A}R^{B}, etc. In some embodiments, R⁸ may be H, CF₃, OH, C₁₋₆ alkyl, or C₁₋₆ alkoxy. In some embodiments, R⁸ may be H.

With respect to any relevant structural representation, such as Formula 2, 3, or 4, Y is a bond, —C(R^{C}R^{D})—, —C(═O)—, —O—, —N(R^{A})—, —S(O)₀₋₂-. In some embodiments, Y is a bond. In some embodiments, Y is —C(R^{C}R^{D})-. In some embodiments, Y is —CH-. In some embodiments, Y is —C(═O)-. In some embodiments, Y is —O-. In some embodiments, Y is —N(R^{A})-. In some embodiments, Y is —N-. In some embodiments, Y is —S(O)O₂-. In some embodiments, Y is —S-. In some embodiments, Y is —SO₂-.

With respect to any relevant structural representation, such as Formula 2, 3, or 4, Z is —C(R^{C}R^{D})—, —C(═O)—, —O—, —N(R^{A})-, or —S(O)₀₋₂-. In some embodiments, Z is —C(R^{C}R^{D})-. In some embodiments, Z is —C(═O)-. In some embodiments, Z is —O -. In some embodiments, Z is —N(R^{A})-. In some embodiments, Z is —N(CH₃)-. In some embodiments, Z is —S(O)₀₋₂-. In some embodiments, Z is —N(CH₃)-. In some embodiments, Z is —CH₂-. In some embodiments, Z is CH. In some embodiments, Z is —CH(CH₃)-. In some embodiments, Z is —C(Br)-.

With respect to any relevant structural representation, such as Formula 2, 3, or 4, W is —C(R^{C}R^{D})—, —C(═O)-, or —SO₂-. In some embodiments, W is —C(R^{C}R^{D})-. In some embodiments, W is —C(═O)-. In some embodiments, W is —SO₂-. In some embodiments, W is —CH(CH₃)-. In some embodiments, W is CH₂.

With respect to any relevant structural representation, such as Formula 2 or 3, in some embodiments, when Ring B is the fused bicyclic heterocyclic ring system, and Y or Z is —C(R^{C}R^{D}), none of Z and Y is —(C═O)-.

With respect to any relevant structural representation, such as Formula 4, in some embodiments, G is N or CR. In some embodiments, G is N.

With respect to any relevant structural representation, such as Formula 1, X is —O—, —CH₂-, or —CF₂-. In some embodiments, X is —CF₂-. In some embodiments, X is —O-. In some embodiments, X is —CH₂-.

With respect to any relevant structural representation, such as Formula 1, L is optionally substituted C₁₋₃ hydrocarbylene (e.g. —CH₂—, —C₂H₄, —CH═CH—, —C₃H₆-), optionally substituted —O-C₁₋₂ hydrocarbylene-(e.g. —O—CH₂—, —O—CH═CH—, —O—C₂H₄-, etc.), optionally substituted —S—C₁₋₂ hydrocarbylene-, or optionally substituted —NR^{A}—C₁₋₂ hydrocarbylene-. In some embodiments, L is C₁₋₃ hydrocarbylene. In some embodiments, L is —O—C₁₋₂ hydrocarbylene-. In some embodiments, L is —S—C₁₋₂ hydrocarbylene-. In some embodiments, L is —NR^{A}-C₁₋₂ hydrocarbylene-. In some embodiments, L is —CH₂—CH₂-. In some embodiments, L is —CH₂—CH₂—CH₂-.

The embodiments in Table 1A below are individually contemplated, wherein any one of the embodiments contains a compound of Formula 1, and the particular Ring A and the particular Ring B identified for each embodiment.

TABLE 1A

| Em- | | |
|---|---|---|
| bodi- | | |
| ment | Ring A | Ring B |
| E1 | optionally substituted Ring A-1 | optionally substituted Ring B-1 |
| E2 | optionally substituted Ring A-1 | optionally substituted Ring B-2 |
| E3 | optionally substituted Ring A-1 | optionally substituted Ring B-3 |
| E4 | optionally substituted Ring A-1 | optionally substituted Ring B-4 |
| E5 | optionally substituted Ring A-1 | optionally substituted Ring B-5 |
| E6 | optionally substituted Ring A-1 | optionally substituted Ring B-6 |
| E7 | optionally substituted Ring A-1 | optionally substituted Ring B-7 |
| E8 | optionally substituted Ring A-1 | optionally substituted Ring B-8 |
| E9 | optionally substituted Ring A-1 | optionally substituted Ring B-9 |
| E10 | optionally substituted Ring A-1 | optionally substituted Ring B-10 |
| E11 | optionally substituted Ring A-1 | optionally substituted Ring B-11 |
| E12 | optionally substituted Ring A-1 | optionally substituted Ring B-12 |
| E13 | optionally substituted Ring A-1 | optionally substituted Ring B-13 |
| E14 | optionally substituted Ring A-1 | optionally substituted Ring B-14 |
| E15 | optionally substituted Ring A-1 | optionally substituted Ring B-15 |
| E16 | optionally substituted Ring A-1 | optionally substituted Ring B-16 |
| E17 | optionally substituted Ring A-1 | optionally substituted Ring B-17 |
| E18 | optionally substituted Ring A-1 | optionally substituted Ring B-18 |
| E19 | optionally substituted Ring A-2 | optionally substituted Ring B-1 |
| E20 | optionally substituted Ring A-2 | optionally substituted Ring B-2 |
| E21 | optionally substituted Ring A-2 | optionally substituted Ring B-3 |
| E22 | optionally substituted Ring A-2 | optionally substituted Ring B-4 |
| E23 | optionally substituted Ring A-2 | optionally substituted Ring B-5 |
| E24 | optionally substituted Ring A-2 | optionally substituted Ring B-6 |
| E25 | optionally substituted Ring A-2 | optionally substituted Ring B-7 |
| E26 | optionally substituted Ring A-2 | optionally substituted Ring B-8 |
| E27 | optionally substituted Ring A-2 | optionally substituted Ring B-9 |
| E28 | optionally substituted Ring A-2 | optionally substituted Ring B-10 |
| E29 | optionally substituted Ring A-2 | optionally substituted Ring B-11 |
| E30 | optionally substituted Ring A-2 | optionally substituted Ring B-12 |
| E31 | optionally substituted Ring A-2 | optionally substituted Ring B-13 |
| E32 | optionally substituted Ring A-2 | optionally substituted Ring B-14 |
| E33 | optionally substituted Ring A-2 | optionally substituted Ring B-15 |
| E34 | optionally substituted Ring A-2 | optionally substituted Ring B-16 |
| E35 | optionally substituted Ring A-2 | optionally substituted Ring B-17 |
| E36 | optionally substituted Ring A-2 | optionally substituted Ring B-18 |
| E37 | optionally substituted Ring A-3 | optionally substituted Ring B-1 |
| E38 | optionally substituted Ring A-3 | optionally substituted Ring B-2 |
| E39 | optionally substituted Ring A-3 | optionally substituted Ring B-3 |
| E40 | optionally substituted Ring A-3 | optionally substituted Ring B-4 |
| E41 | optionally substituted Ring A-3 | optionally substituted Ring B-5 |
| E42 | optionally substituted Ring A-3 | optionally substituted Ring B-6 |
| E43 | optionally substituted Ring A-3 | optionally substituted Ring B-7 |
| E44 | optionally substituted Ring A-3 | optionally substituted Ring B-8 |
| E45 | optionally substituted Ring A-3 | optionally substituted Ring B-9 |
| E46 | optionally substituted Ring A-3 | optionally substituted Ring B-10 |
| E47 | optionally substituted Ring A-3 | optionally substituted Ring B-11 |
| E48 | optionally substituted Ring A-3 | optionally substituted Ring B-12 |
| E49 | optionally substituted Ring A-3 | optionally substituted Ring B-13 |
| E50 | optionally substituted Ring A-3 | optionally substituted Ring B-14 |
| E51 | optionally substituted Ring A-3 | optionally substituted Ring B-15 |
| E52 | optionally substituted Ring A-3 | optionally substituted Ring B-16 |
| E53 | optionally substituted Ring A-3 | optionally substituted Ring B-17 |
| E54 | optionally substituted Ring A-3 | optionally substituted Ring B-18 |
| E55 | optionally substituted Ring A-4 | optionally substituted Ring B-1 |
| E56 | optionally substituted Ring A-4 | optionally substituted Ring B-2 |
| E57 | optionally substituted Ring A-4 | optionally substituted Ring B-3 |
| E58 | optionally substituted Ring A-4 | optionally substituted Ring B-4 |
| E59 | optionally substituted Ring A-4 | optionally substituted Ring B-5 |
| E60 | optionally substituted Ring A-4 | optionally substituted Ring B-6 |
| E61 | optionally substituted Ring A-4 | optionally substituted Ring B-7 |
| E62 | optionally substituted Ring A-4 | optionally substituted Ring B-8 |
| E63 | optionally substituted Ring A-4 | optionally substituted Ring B-9 |
| E64 | optionally substituted Ring A-4 | optionally substituted Ring B-10 |
| E65 | optionally substituted Ring A-4 | optionally substituted Ring B-11 |
| E66 | optionally substituted Ring A-4 | optionally substituted Ring B-12 |
| E67 | optionally substituted Ring A-4 | optionally substituted Ring B-13 |
| E68 | optionally substituted Ring A-4 | optionally substituted Ring B-14 |
| E69 | optionally substituted Ring A-4 | optionally substituted Ring B-15 |
| E70 | optionally substituted Ring A-4 | optionally substituted Ring B-16 |
| E71 | optionally substituted Ring A-4 | optionally substituted Ring B-17 |
| E72 | optionally substituted Ring A-4 | optionally substituted Ring B-18 |
| E73 | optionally substituted Ring A-5 | optionally substituted Ring B-1 |
| E74 | optionally substituted Ring A-5 | optionally substituted Ring B-2 |
| E75 | optionally substituted Ring A-5 | optionally substituted Ring B-3 |
| E76 | optionally substituted Ring A-5 | optionally substituted Ring B-4 |
| E77 | optionally substituted Ring A-5 | optionally substituted Ring B-5 |
| E78 | optionally substituted Ring A-5 | optionally substituted Ring B-6 |
| E79 | optionally substituted Ring A-5 | optionally substituted Ring B-7 |
| E80 | optionally substituted Ring A-5 | optionally substituted Ring B-8 |
| E81 | optionally substituted Ring A-5 | optionally substituted Ring B-9 |
| E82 | optionally substituted Ring A-5 | optionally substituted Ring B-10 |
| E83 | optionally substituted Ring A-5 | optionally substituted Ring B-11 |
| E84 | optionally substituted Ring A-5 | optionally substituted Ring B-12 |
| E85 | optionally substituted Ring A-5 | optionally substituted Ring B-13 |
| E86 | optionally substituted Ring A-5 | optionally substituted Ring B-14 |
| E87 | optionally substituted Ring A-5 | optionally substituted Ring B-15 |
| E88 | optionally substituted Ring A-5 | optionally substituted Ring B-16 |
| E89 | optionally substituted Ring A-5 | optionally substituted Ring B-17 |
| E90 | optionally substituted Ring A-5 | optionally substituted Ring B-18 |
| E91 | optionally substituted Ring A-6 | optionally substituted Ring B-1 |
| E92 | optionally substituted Ring A-6 | optionally substituted Ring B-2 |
| E93 | optionally substituted Ring A-6 | optionally substituted Ring B-3 |
| E94 | optionally substituted Ring A-6 | optionally substituted Ring B-4 |
| E95 | optionally substituted Ring A-6 | optionally substituted Ring B-5 |
| E96 | optionally substituted Ring A-6 | optionally substituted Ring B-6 |
| E97 | optionally substituted Ring A-6 | optionally substituted Ring B-7 |
| E98 | optionally substituted Ring A-6 | optionally substituted Ring B-8 |
| E99 | optionally substituted Ring A-6 | optionally substituted Ring B-9 |
| E100 | optionally substituted Ring A-6 | optionally substituted Ring B-10 |
| E101 | optionally substituted Ring A-6 | optionally substituted Ring B-11 |
| E102 | optionally substituted Ring A-6 | optionally substituted Ring B-12 |
| E103 | optionally substituted Ring A-6 | optionally substituted Ring B-13 |
| E104 | optionally substituted Ring A-6 | optionally substituted Ring B-14 |
| E105 | optionally substituted Ring A-6 | optionally substituted Ring B-15 |
| E106 | optionally substituted Ring A-6 | optionally substituted Ring B-16 |
| E107 | optionally substituted Ring A-6 | optionally substituted Ring B-17 |
| E108 | optionally substituted Ring A-6 | optionally substituted Ring B-18 |

For some embodiments of Table 1A, L is —CH₂CH₂-.

Some embodiments include optionally substituted 7-(2-((1S,2R,3S,4R)-2,3-dihydroxy-4-(7H-pyrrolo[2,3-d]pyrimidin-7-yl)cyclopenty)ethyl)quinazolin-4(3H)-one.

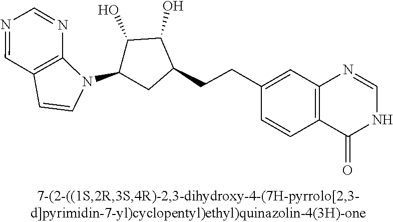 [SMILES: O=c1[nH]cnc2cc(CC[C@H]3C[C@@H](n4ccc5cncnc54)[C@H](O)[C@@H]3O)ccc12]

Some embodiments include optionally substituted (1S,2R,3S,5R)-3-(2-(3,4-dihydro-2H-[1,4]oxazino[3,2-b]quinolin-7-ypethyl)-5-(7H-pyrrolo[2,3-d]pyrimidin-7-yl)cyclopentane-1,2-diol.

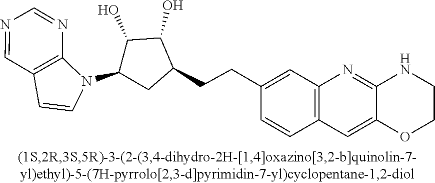 [SMILES: O[C@@H]1[C@@H](CCc2ccc3cc4c(nc3c2)NCCO4)C[C@@H](n2ccc3cncnc32)[C@@H]1O]

Some embodiments include optionally substituted 7-(2-((1S,2R,3S,4R)-2,3-dihydroxy-4-(7H-pyrrolo[2,3-d]pyrimidin-7-yl)cyclopentyl)ethyl)-2H-[1,4]oxazino[3,2-b]quinolin-3(4H)-one.

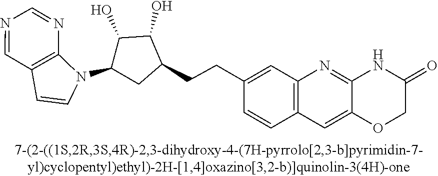 [SMILES: O=C1COc2cc3ccc(CC[C@H]4C[C@@H](n5ccc6cncnc65)[C@H](O)[C@@H]4O)cc3nc2N1]

Some embodiments include optionally substituted (1S,2R,3S,5R)-3-(2-(3,4-dihydro-2H-[1,4]thiazino[3,2-b]quinolin-7-ypethyl)-5-(7H-pyrrolo[2,3-d]pyrimidin-7-yl)cyclopentane-1,2-diol.

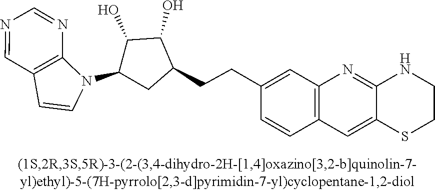 [SMILES: O[C@@H]1[C@@H](CCc2ccc3cc4c(nc3c2)NCCS4)C[C@@H](n2ccc3cncnc32)[C@@H]1O]

Some embodiments include optionally substituted 6-(2-((1S,2R,3S,4R)-2,3-dihydroxy-4-(7H-pyrrolo[2,3-d]pyrimidin-7-yl)cyclopentyl)ethyl)-2H-benzo[e][1,2,4]thiadiazine 1,1-dioxide.

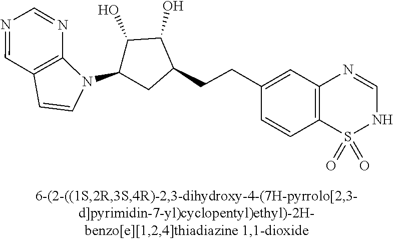 [SMILES: O=S1(=O)NC=Nc2cc(CC[C@H]3C[C@@H](n4ccc5cncnc54)[C@H](O)[C@@H]3O)ccc21]

Some embodiments include optionally substituted (1R,2S,3R,5S)-3-(7H-pyrrolo[2,3-d]pyrimidin-7-yl)-5-(3-(quinolin-7-yl)propyl)cyclopentane-1,2-diol.

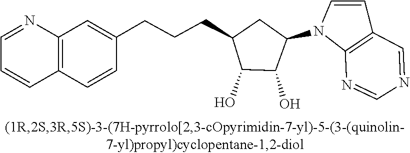 [SMILES: O[C@@H]1[C@@H](CCCc2ccc3cccnc3c2)C[C@@H](n2ccc3cncnc32)[C@@H]1O]

Some embodiments include optionally substituted 7-(2-((1S,2R,3S,4R)-2,3-dihydroxy-4-(7H-pyrrolo[2,3-d]pyrimidin-7-yl)cyclopentyl)ethyl)-2H-[1,4]thiazino[3,2-b]quinolin-3(4H)-one.

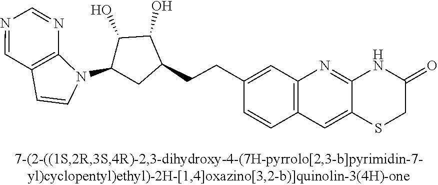 [SMILES: O=C1CSc2cc3ccc(CC[C@H]4C[C@@H](n5ccc6cncnc65)[C@H](O)[C@@H]4O)cc3nc2N1]

Some embodiments include optionally substituted 6-(2-((2R,3S,4R,5R)-3,4-dihydroxy-5-(7H-pyrrolo[2,3-d]pyrimidin-7-yl)tetrahydrofuran-2-ypethyl)-2H-benzo[e][1,2,4]thiadiazine 1,1 -dioxide.

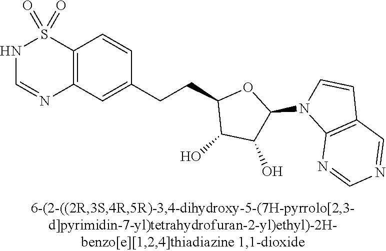 [SMILES: O=S1(=O)NC=Nc2cc(CC[C@H]3O[C@@H](n4ccc5cncnc54)[C@H](O)[C@@H]3O)ccc21]

Some embodiments include optionally substituted 8-(2-((1S,2R,3S,4R)-2,3-dihydroxy-4-(7H-pyrrolo[2,3-d]pyrimidin-7-yl)cyclopentyl)ethyl)-2,3- dihydroimidazo[2,1-b]quinazolin-5(1H)-one.

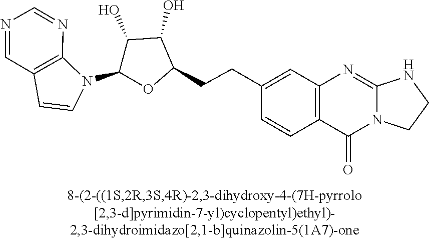 [SMILES: O=c1c2ccc(CC[C@H]3O[C@@H](n4ccc5cncnc54)[C@H](O)[C@@H]3O)cc2nc2n1CCN2]

Some embodiments include optionally substituted (2R,3S,4R,5R)-2-(2-(2H-benzo[b][1,4]oxazin-6-yl)ethyl)-5-(7H-pyrrolo[2,3-d]pyrimidin-7- yl)tetrahydrofuran-3,4-diol.

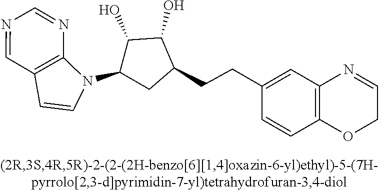 [SMILES: O[C@@H]1[C@@H](CCc2ccc3c(c2)N=CCO3)C[C@@H](n2ccc3cncnc32)[C@@H]1O]

Some embodiments include optionally substituted 6-(2-((1S,2R,3S,4R)-2,3-dihydroxy-4-(7H-pyrrolo[2,3-d]pyrimidin-7-yl)cyclopentyl)ethyl)-2H-benzo[b][1,4]thiazine 1,1-dioxide.

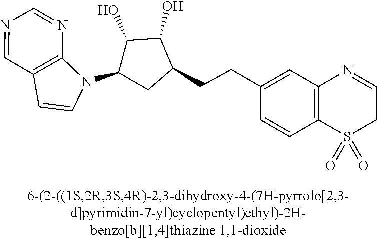 [SMILES: O=S1(=O)CC=Nc2cc(CC[C@H]3C[C@@H](n4ccc5cncnc54)[C@H](O)[C@@H]3O)ccc21]

Some embodiments include one of the compounds below:

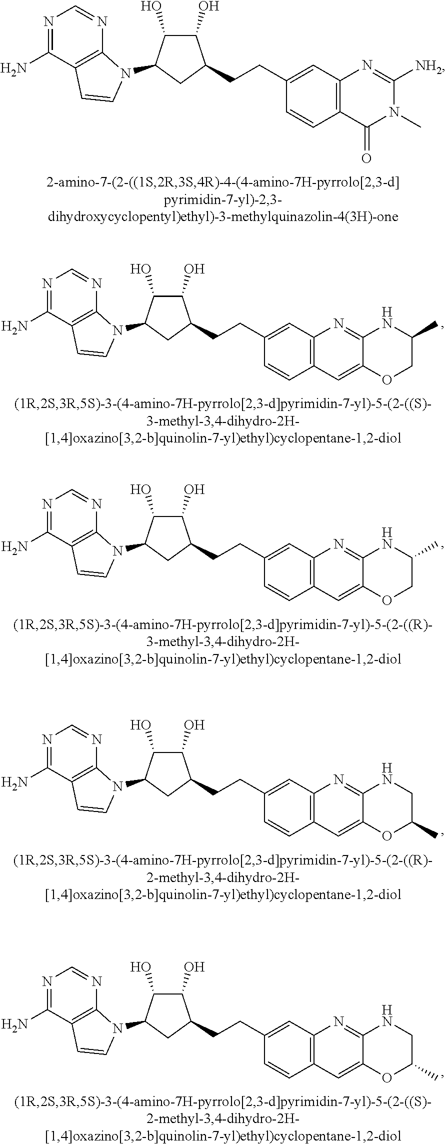 [SMILES: Cn1c(N)nc2cc(CC[C@H]3C[C@@H](n4ccc5c(N)ncnc54)[C@H](O)[C@@H]3O)ccc2c1=O ; C[C@H]1COc2cc3ccc(CC[C@H]4C[C@@H](n5ccc6c(N)ncnc65)[C@H](O)[C@@H]4O)cc3nc2N1 ; C[C@@H]1COc2cc3ccc(CC[C@H]4C[C@@H](n5ccc6c(N)ncnc65)[C@H](O)[C@@H]4O)cc3nc2N1 ; C[C@@H]1CNc2nc3cc(CC[C@H]4C[C@@H](n5ccc6c(N)ncnc65)[C@H](O)[C@@H]4O)ccc3cc2O1 ; C[C@H]1CNc2nc3cc(CC[C@H]4C[C@@H](n5ccc6c(N)ncnc65)[C@H](O)[C@@H]4O)ccc3cc2O1]

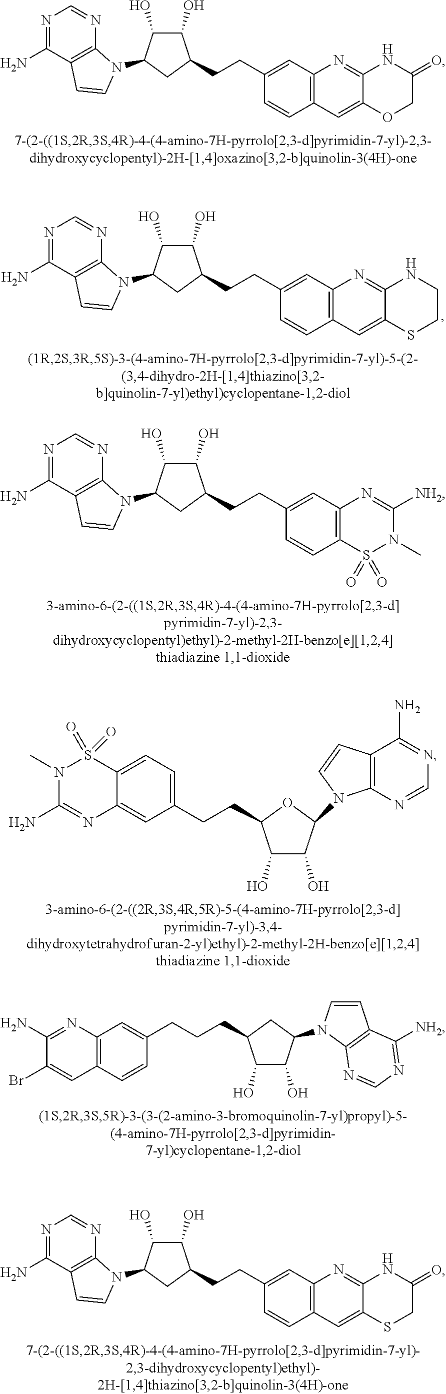 [SMILES: Nc1ncnc2c1ccn2[C@@H]1C[C@H](CCc2ccc3cc4c(nc3c2)NC(=O)CO4)[C@@H](O)[C@H]1O ; Nc1ncnc2c1ccn2[C@@H]1C[C@H](CCc2ccc3cc4c(nc3c2)NCCS4)[C@@H](O)[C@H]1O ; CN1C(N)=Nc2cc(CC[C@H]3C[C@@H](n4ccc5c(N)ncnc54)[C@H](O)[C@@H]3O)ccc2S1(=O)=O ; CN1C(N)=Nc2cc(CC[C@H]3O[C@@H](n4ccc5c(N)ncnc54)[C@H](O)[C@@H]3O)ccc2S1(=O)=O ; Nc1nc2cc(CCC[C@H]3C[C@@H](n4ccc5c(N)ncnc54)[C@H](O)[C@@H]3O)ccc2cc1Br ; Nc1ncnc2c1ccn2[C@@H]1C[C@H](CCc2ccc3cc4c(nc3c2)NC(=O)CS4)[C@@H](O)[C@H]1O]

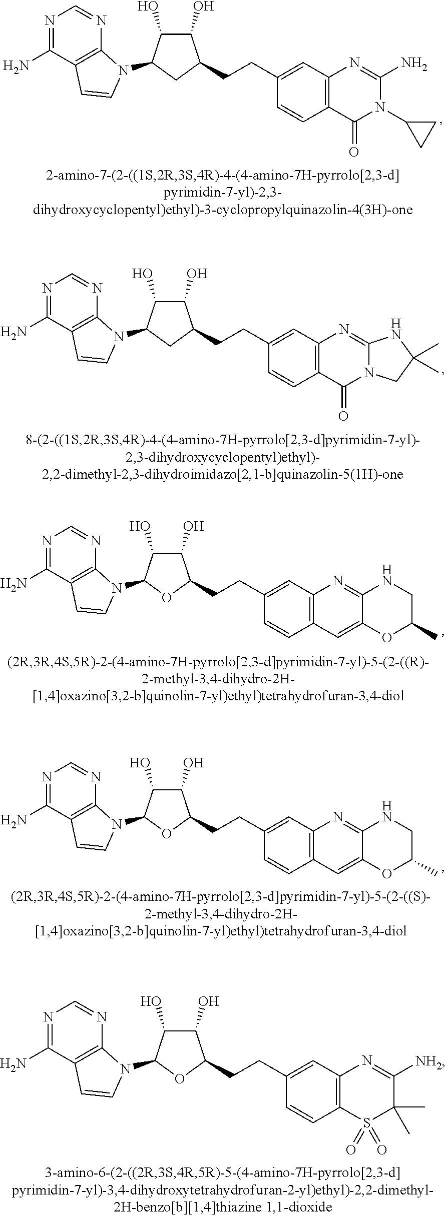 [SMILES: Nc1ncnc2c1ccn2[C@@H]1C[C@H](CCc2ccc3c(=O)n(C4CC4)c(N)nc3c2)[C@@H](O)[C@H]1O ; CC1(C)Cn2c(nc3cc(CC[C@H]4C[C@@H](n5ccc6c(N)ncnc65)[C@H](O)[C@@H]4O)ccc3c2=O)N1 ; C[C@@H]1CNc2nc3cc(CC[C@H]4O[C@@H](n5ccc6c(N)ncnc65)[C@H](O)[C@@H]4O)ccc3cc2O1 ; C[C@H]1CNc2nc3cc(CC[C@H]4O[C@@H](n5ccc6c(N)ncnc65)[C@H](O)[C@@H]4O)ccc3cc2O1 ; CC1(C)C(N)=Nc2cc(CC[C@H]3O[C@@H](n4ccc5c(N)ncnc54)[C@H](O)[C@@H]3O)ccc2S1(=O)=O]

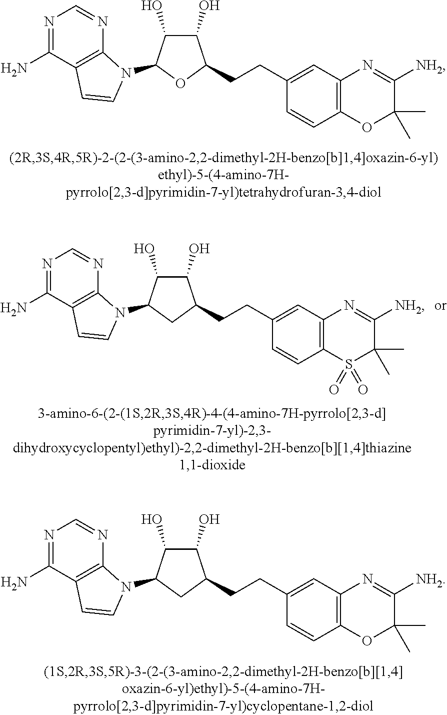 [SMILES: CC1(C)Oc2ccc(CC[C@H]3O[C@@H](n4ccc5c(N)ncnc54)[C@H](O)[C@@H]3O)cc2N=C1N ; CC1(C)C(N)=Nc2cc(CC[C@H]3C[C@@H](n4ccc5c(N)ncnc54)[C@H](O)[C@@H]3O)ccc2S1(=O)=O ; CC1(C)Oc2ccc(CC[C@H]3C[C@@H](n4ccc5c(N)ncnc54)[C@H](O)[C@@H]3O)cc2N=C1N]

Some embodiments include one of the compounds listed in Table 1B below, wherein each structure can be optionally substituted.

TABLE 1B
Compound structures and their ID numbers
Compound Structure and ID Number

| | |
|---|---|
| 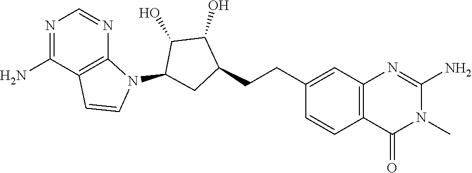 [SMILES: Cn1c(N)nc2cc(CC[C@H]3C[C@@H](n4ccc5c(N)ncnc54)[C@H](O)[C@@H]3O)ccc2c1=O] | 1-7 |
| 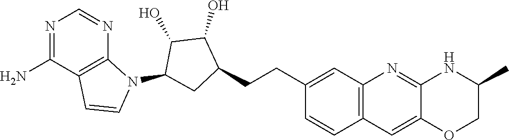 [SMILES: C[C@H]1COc2cc3ccc(CC[C@H]4C[C@@H](n5ccc6c(N)ncnc65)[C@H](O)[C@@H]4O)cc3nc2N1] | 1-8 |
| 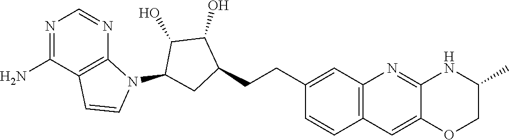 [SMILES: C[C@@H]1COc2cc3ccc(CC[C@H]4C[C@@H](n5ccc6c(N)ncnc65)[C@H](O)[C@@H]4O)cc3nc2N1] | 1-9 |
| 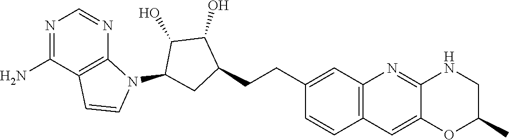 [SMILES: C[C@@H]1CNc2nc3cc(CC[C@H]4C[C@@H](n5ccc6c(N)ncnc65)[C@H](O)[C@@H]4O)ccc3cc2O1] | 1-10 |
| 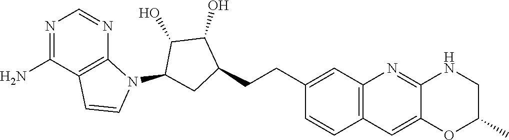 [SMILES: C[C@H]1CNc2nc3cc(CC[C@H]4C[C@@H](n5ccc6c(N)ncnc65)[C@H](O)[C@@H]4O)ccc3cc2O1] | 1-11 |
| 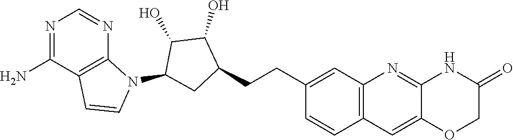 [SMILES: Nc1ncnc2c1ccn2[C@@H]1C[C@H](CCc2ccc3cc4c(nc3c2)NC(=O)CO4)[C@@H](O)[C@H]1O] | 1-12 |
| 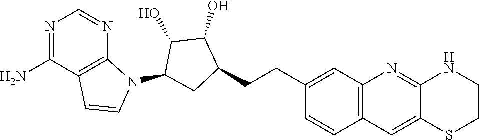 [SMILES: Nc1ncnc2c1ccn2[C@@H]1C[C@H](CCc2ccc3cc4c(nc3c2)NCCS4)[C@@H](O)[C@H]1O] | 1-13 |
| 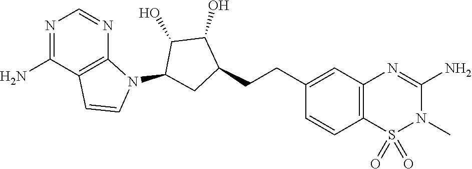 [SMILES: CN1C(N)=Nc2cc(CC[C@H]3C[C@@H](n4ccc5c(N)ncnc54)[C@H](O)[C@@H]3O)ccc2S1(=O)=O] | 1-14 |
| 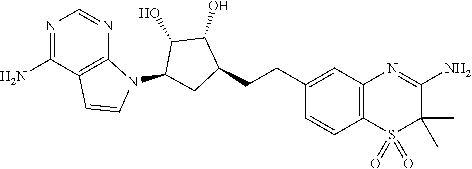 [SMILES: CC1(C)C(N)=Nc2cc(CC[C@H]3C[C@@H](n4ccc5c(N)ncnc54)[C@H](O)[C@@H]3O)ccc2S1(=O)=O] | 1-15 |
| 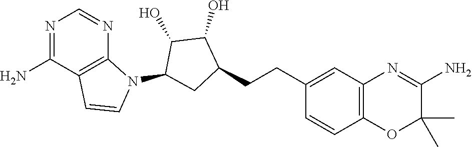 [SMILES: CC1(C)Oc2ccc(CC[C@H]3C[C@@H](n4ccc5c(N)ncnc54)[C@H](O)[C@@H]3O)cc2N=C1N] | 1-16 |
| 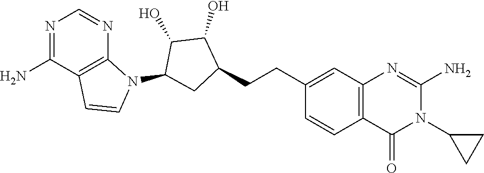 [SMILES: Nc1ncnc2c1ccn2[C@@H]1C[C@H](CCc2ccc3c(=O)n(C4CC4)c(N)nc3c2)[C@@H](O)[C@H]1O] | 1-17 |
| 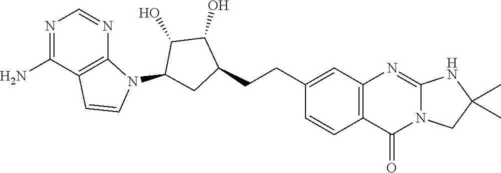 [SMILES: CC1(C)Cn2c(nc3cc(CC[C@H]4C[C@@H](n5ccc6c(N)ncnc65)[C@H](O)[C@@H]4O)ccc3c2=O)N1] | 1-18 |
| 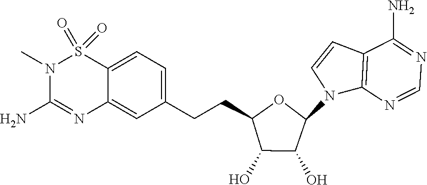 [SMILES: CN1C(N)=Nc2cc(CC[C@H]3O[C@@H](n4ccc5c(N)ncnc54)[C@H](O)[C@@H]3O)ccc2S1(=O)=O] | 2-8 |
| 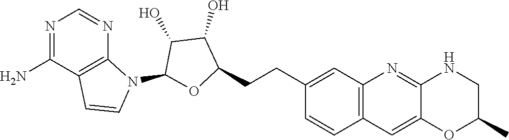 [SMILES: C[C@@H]1CNc2nc3cc(CC[C@H]4O[C@@H](n5ccc6c(N)ncnc65)[C@H](O)[C@@H]4O)ccc3cc2O1] | 2-9 |
| 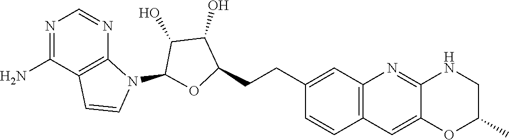 [SMILES: C[C@H]1CNc2nc3cc(CC[C@H]4O[C@@H](n5ccc6c(N)ncnc65)[C@H](O)[C@@H]4O)ccc3cc2O1] | 2-10 |
| 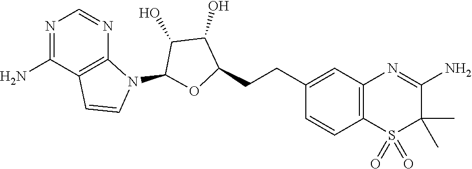 [SMILES: CC1(C)C(N)=Nc2cc(CC[C@H]3O[C@@H](n4ccc5c(N)ncnc54)[C@H](O)[C@@H]3O)ccc2S1(=O)=O] | 2-11 |
| 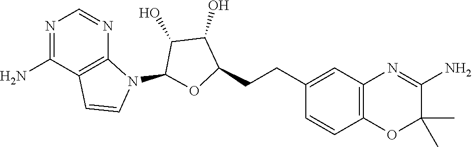 [SMILES: CC1(C)Oc2ccc(CC[C@H]3O[C@@H](n4ccc5c(N)ncnc54)[C@H](O)[C@@H]3O)cc2N=C1N] | 2-12 |
| 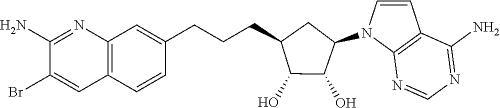 [SMILES: Nc1nc2cc(CCC[C@H]3C[C@@H](n4ccc5c(N)ncnc54)[C@H](O)[C@@H]3O)ccc2cc1Br] | 3-7 |
| 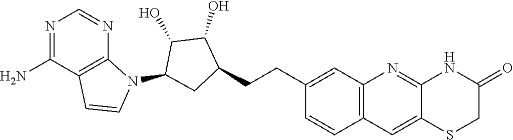 [SMILES: Nc1ncnc2c1ccn2[C@@H]1C[C@H](CCc2ccc3cc4c(nc3c2)NC(=O)CS4)[C@@H](O)[C@H]1O] | 4-3 |

Some embodiments include use of a compound described herein, such as a compound of Formula 1, optionally substituted 7-(2-((1S,2R,3S,4R)-2,3-dihydroxy-4-(7H-pyrrolo[2,3-d]pyrimidin-7-yl)cyclopentyl)ethyl)quinazolin-4(3H)- one, optionally substituted (1 S,2R,3S,5R)-3-(2-(3,4-dihydro-2H-[1,4]oxazino[3,2-b]quinolin-7-ypethyl)-5-(7H-pyrrolo[2,3-d]pyrimidin-7-yl)cyclopentane-1,2-diol, optionally substituted 7-(2-((1S,2R,3S,4R)-2,3-dihydroxy-4-(7H-pyrrolo[2,3-d]pyrimidin-7-yl)cyclopentyl)ethyl)-2H-[1,4]oxazino[3,2-b]quinolin-3(4H)-one, optionally substituted (1S,2R,3S,5R)-3-(2-(3,4-dihydro-2H-[1,4]thiazino[3,2-b]quinolin-7-yl)ethyl)-5-(7H-pyrrolo[2,3-d]pyrimidin-7-yl)cyclopentane-1,2-diol, optionally substituted 6-(2-((1 S,2R,3S,4R)-2,3-dihydroxy-4-(7H-pyrrolo[2,3-d]pyrimidin-7-yl)cyclopentyl)ethyl)-2H-benzo[e][1,2,4]thiadiazine 1,1-dioxide, optionally substituted (1 R,2S,3R,5S)-3-(7H-pyrrolo[2,3-d]pyrimidin-7-yl)-5-(3-(quinolin-7-yl)propyl)cyclopentane-1,2- diol, optionally substituted 7-(2-((1S,2R,3S,4R)-2,3-dihydroxy-4-(7H-pyrrolo[2,3-d]pyrimidin-7-yl)cyclopentyl)ethyl)-2H-[1,4]thiazino[3,2-b]quinolin-3(4H)-one, optionally substituted 6-(2-((2R,3S,4R,5R)-3,4-dihydroxy-5-(7H-pyrrolo[2,3-d]pyrimidin-7-yl)tetrahydrofuran-2-ypethyl)-2H-benzo[e][1,2,4]thiadiazine 1,1-dioxide, optionally substituted 8-(2-((1S,2R,3S,4R)-2,3-dihydroxy-4-(7H-pyrrolo[2,3-d]pyrimidin-7-yl)cyclopentyl)ethyl)-2,3-dihydroimidazo[2,1 -b]quinazolin-5(1 H)-one, optionally substituted (2R,3S,4R,5R)-2-(2-(2H-benzo[b][1 ,4]oxazin-6-ypethyl)-5-(7H-pyrrolo[2,3-d]pyrimidin-7-yl)tetrahydrofuran-3,4-diol, optionally substituted 6-(2-((1S,2R,3S,4R)-2,3-dihydroxy-4-(7H-pyrrolo[2,3-d]pyrimidin-7-yl)cyclopentyl)ethyl)-2H-benzo[b][1,4]thiazine 1,1- dioxide, or any compound described herein, or a pharmaceutically acceptable salt thereof, in the manufacture of a medicament for the treatment of cancer, infectious diseases, and other PRMT5 related disorders.

A pharmaceutical composition comprising a subject compound may be adapted for oral, or parental, such as intravenous, intramuscular, topical, intraperitoneal, nasal, buccal, sublingual, or subcutaneous administration, or for administration via respiratory tract in the form of, for example, an aerosol or an air-suspended fine powder. The dosage of a subject compound may vary depending on the route of administration, body weight, age, the type and condition of the disease being treated. A pharmaceutical composition provided herein may optionally comprise two or more subject compounds without an additional therapeutic agent, or may comprise an additional therapeutic agent (i.e., a therapeutic agent other than a compound provided herein). For example, the compounds of the disclosure can be used in combination with at least one other therapeutic agent. Therapeutic agents include, but are not limited to antibiotics, antiemetic agents, antidepressants, and antifungal agents, anti-inflammatory agents, antiviral agents, and anticancer agents that are known in the art. The pharmaceutical composition may be used for the treatment of cancer, and other PRMT5-related diseases or disorders in patients. The term “patient” herein means a mammal (e.g., a human or an animal). In some embodiments, the patient has cancer.

The pharmaceutical composition comprising a subject compound can be prepared by combining a subject compound with at least one pharmaceutical acceptable inert ingredient, such as a carrier, excipient, filler, lubricant, flavoring agent, buffer, etc., selected on the basis of the chosen route of administration and standard pharmaceutical practice as described, for example, in Remington's Pharmaceutical Sciences, 2005, the disclosure of which is hereby incorporated herein by reference, in its entirety. The relative proportions of active ingredient and carrier may be determined, for example, by the solubility and chemical nature of the compounds, chosen route of administration and standard pharmaceutical practice.

Some embodiments include a method of treating a disease or disorder associated with PRMT5 comprising administering a therapeutically effective amount of a subject compound or a pharmaceutical composition comprising a subject compound to a patient in need thereof. The term a “therapeutically effective amount” herein refers to an amount of a subject compound or a pharmaceutical composition of the present disclosure provided herein sufficient to be effective in inhibiting PRMT5 enzyme and thus providing a benefit in the treatment of cancer, infectious diseases, and other PRMT5 associated disorders, to delay or minimize symptoms associated with cancer, infectious diseases, and other PRMT5 associated disorders, or to ameliorate a disease or infection or cause thereof (e.g. 0.1-1000 mg). The term “treatment” refers to causing a therapeutically beneficial effect, such as ameliorating existing symptoms, ameliorating the underlying causes of symptoms, postponing, preventing the further development of a disorder, or reducing the severity of symptoms that are otherwise expected to develop without treatment.

### Experimental Section

### Preparation of Compounds

The compounds of the disclosure can be made using procedures known in the art. The following reaction schemes show typical procedures, but those skilled in the art will recognize that other procedures can also be suitable for using to prepare these compounds. For examples in Formula I and II, wherein R¹ is not hydrogen, those skilled in the art will recognize that changes to the requisite reagents can be made at the appropriate steps in the synthetic methods outlined below. Reactions may involve monitoring for consumption of starting materials, and there are many methods for the monitoring, including but not limited to thin layer chromatography (TLC) and liquid chromatography mass spectrometry (LCMS). Those skilled in the art will recognize that any synthetic method specified in the examples shown below can be substituted by other non-limiting methods when suitable.

Some of the techniques, solvents and reagents can be referred to by their abbreviations as follows:
Acetonitrile: MeCN or ACN
Aqueous: aq.
Benzyl: Bn
9-Borabicyclo [3.3.1] nonane: 9-BBN
1-[Bis(dimethylamino)methylene]-1H-1,2,3-triazolo[4,5-b]pyridinium 3-oxid hexafluorophosphate: HATU
N,O-Bis(trimethylsilyl)acetamide: BSA
[1,1′-Bis(diphenylphosphino)ferrocene]-dichloropalladium (II): Pd(dppf)Cl₂
m-CPBA: meta-chloroperoxybenzoic acid
DBTCE: 1,2-dibromotetracholoroethane
2,3-Dichloro-5,6-dicyano-1,4-benzoquinone: DDQ
Dichloromethane: DCM
Diisopropylazodiacarboxylate: DIAD
Diisopropylethylamine: DIPEA, DIEA or iPr₂NEt
Dimethylformamide: DMF
Dimethylsulfoxide: DMSO
1-Ethyl-3-(3-dimethylaminopropyl)carbodiimide: EDCI
Equivalents: equiv.
Ether or diethyl ether: Et₂O
Ethyl acetate: AcOEt or EtOAc
Example: Ex. or ex.
Formic acid: FA
Grams: g
High performance liquid chromatography: HPLC
Hydroxybenzotriazole: HOBT
2-lodoxybenzoic acid: IBX
Inhibition: Inh.
Liquid chromatography mass spectrometry: LCMS or LC-MS
Lithium aluminum hydride: LAH
Lithium hexamethyldisilazide: LiHMDS
Methansulfonyl chloride: MeSO₂Cl
Methyl iodide: Mel
Methanol: MeOH
Microliter: μl
Micrometer: μm
Milligram: mg
Milliliter: mL
Millimole: mmol
(R)-(−)-(3,5-Dioxa-4-phospha-cyclohepta[2,1-a;3,4-a′]dinaphthalen-4-yl)dimethylamine:
(R)-MonoPhos
N-Bromosuccinimide: NBS
n-Butyllithium: n-BuLi
Nuclear magnetic resonance spectroscopy: NMR
Palladium tetra-triphenylphosphine: Pd(PPh₃)₄
N-Phenyl bis(trifluoromethanwsulfonimide): PhNTf₂
Retentional time: tR
Acetylacetonatobis(ethylene)rhodium(I): Rh(acac)(eth)₂
Room temperature (ambient, −25° C.): rt or RT
Potassium tert-butoxide: t-BuOK
Preparative HPLC: Prep-HPLC
Preparative TLC: Prep-TLC
Sodium hydride: NaH
Supercritical Fluid Chromatography: SFC
Tris(2-carboxymethyl)phosphine: TCEP
Temperature: temp.
Tetrahydrofuran: THF
Thin layer chromatography: TLC
Triethylamine: Et₃N or TEA
Tribromoborane: BBr₃
Trifluoroacetic acid: TFA
Trifluoromethanesulfonic anhydride: Tf₂O
Trimethylsilyl trifluoro methanesulfonate: TMSOTf

In the synthetic schemes described below, unless otherwise indicated all temperatures are set forth in degrees Celsius and all parts and percentages are by weight. Reagents and solvents were purchased from commercial suppliers such as Aldrich Chemical Company and were used without further purification unless otherwise indicated. Tetrahydrofuran (THF) and N,N-dimethylformamide (DMF) were purchased from commercial sources in Sure Seal bottles and used as received.

The reactions set forth below were done generally under a positive pressure of argon or nitrogen at an ambient temperature (unless otherwise stated) in anhydrous solvents. Glassware was oven dried and/or heat dried. The reactions were assayed by TLC and/or analyzed by LC-MS and terminated as judged by the consumption of starting material. Analytical thin layer chromatography (TLC) was performed on glass plates pre-coated with silica gel 60 F254 0.25 mm plates (EM Science), and visualized with UV light (254 nm) and/ or heating with commercial ethanolic phosphomolybdic acid. preparative thin layer chromatography (TLC) was performed on glass-plates pre-coated with silica gel 60 F254 0.5 mm plates (20×20 cm, from commercial sources) and visualized with UV light (254 nm).

Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or Mg₂SO₄ prior to filtration and evaporation of the solvents under reduced pressure on a rotary evaporator and noted as solvents removed in vacuo. Column chromatography was completed under positive pressure using 230-400 mesh silica gel.

¹H-NMR spectra and ¹³C-NMR were recorded on a Varian Mercury-VX400 instrument operating at 400 MHZ. NMR spectra were obtained as CDCl₃ solutions (reported in ppm), using chloroform as the reference standard (7.27 ppm for the proton and 77.00 ppm for carbon), CD₃OD (3.4 and 4.8 ppm for the protons and 49.3 ppm for carbon), DMSO-d₆ (2.49 ppm for proton), or internally tetramethylsilane (0.00 ppm) when appropriate. Other NMR solvents were used as needed.

Some of the typical synthetic methods are described in the examples shown below.

### Method 1:

### EXAMPLE 1

### Synthesis of 2-amino-7-(2-((1S,2R,3S,4R)-4-(4-amino-7H-pyrrolo[2,3-d]pyrimidin-7-yl)-2,3-dihydroxycyclopentyl)ethyl)-3-methylquinazolin- 4(3H)-one

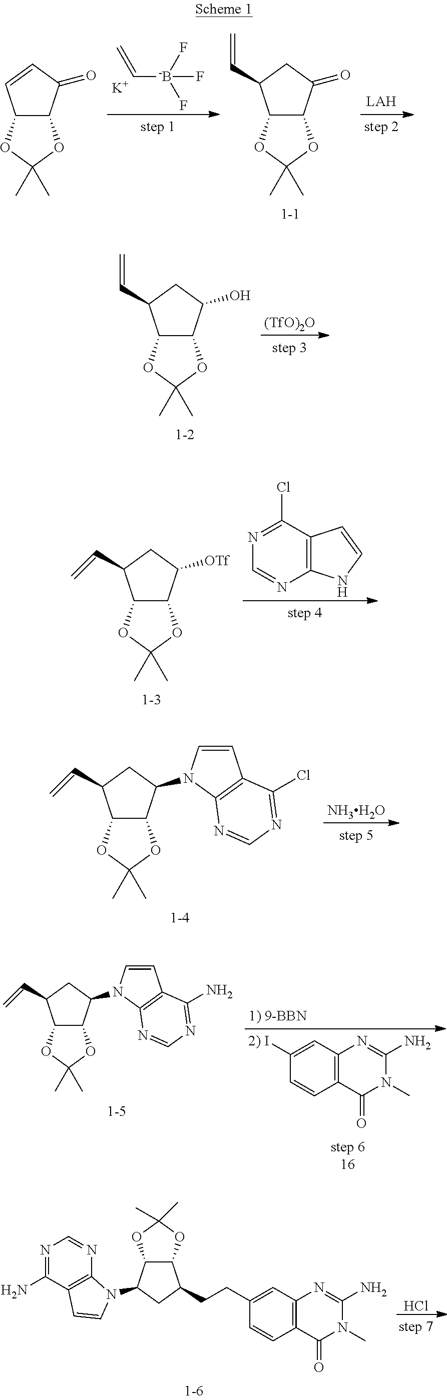

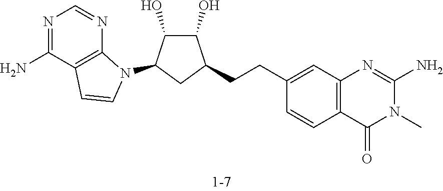 [SMILES: Cn1c(N)nc2cc(CC[C@H]3C[C@@H](n4ccc5c(N)ncnc54)[C@H](O)[C@@H]3O)ccc2c1=O]

Step 1: Synthesis of (3aR,6R,6aR)-2,2-dimethyl-6-vinyltetrahydro-4H-cyclopenta[d][1,3]dioxol-4-one

To a stirred solution of 0.34 g (1.30 mmol) of Rh(acac)(eth)2 and 1.17 g (3.24 mmol) of (R)-MonoPhos in 200 mL of ethanol were added 10.0 g (64.94 mmol) of (3aR,6aR)-2,2-dimethyl-3a,6a-dihydro-4H-cyclopenta[d][1,3]dioxol-4-one and 17.4 g (129.85 mmol) of potassium ethenyltrifluoroborate. The mixture was stirred at 80° C. for 2 h under N₂ atmosphere and filtered; the filter cake was washed with three 30 mL portions of ethanol. The combined filtrates were concentrated; the residue was diluted with 50 mL of water, and extracted with three 50 mL portions of ethyl acetate. The combined organic extracts were washed with brine, and dried over anhydrous Na₂SO₄. After filtration, the filtrate was concentrated to afford a residue, which was purified by chromatography on silica gel column eluting with 0 to 3% gradient of ethyl acetate in petroleum ether to afford compound 1-1. ¹H NMR (400 MHz, CDCl₃) δ 5.84 (ddd, J=17.2, 10.6, 6.4 Hz, 1 H), 5.25-5.06 (m, 2 H), 4.65 (dt, J=5.4, 1.2 Hz, 1 H), 4.21 (dd, J=5.2, 0.8 Hz, 1 H), 3.18-3.07 (m, 1 H), 2.85 (ddd, J=18.3, 8.6, 1.0 Hz, 1 H), 2.38-2.25 (m, 1 H), 1.48-1.44 (m, 3 H), 1.36 (d, J=0.7 Hz, 3 H).

### Step 2:

To a stirred solution of 18.7 mL (18.7 mmol, 1 M in THF) of lithium aluminumhydride in 60 mL THF was added 8.5 g (46.7 mmol) of compound 1-1 dropwise at −78 ° C. The mixture was stirred at −78° C. for 1 h and then quenched by addition of 0.7 mL of water, 0.7 mL of 15% NaOH solution and 2.1 mL of water at −78 ° C. The resulting mixture was filtered; the filter cake was washed with three 50 mL portions of ethyl acetate. The combined filtrates were dried over anhydrous Na₂SO₄. After filtration, the filtrate was concentrated to afford a residue, which was purified by chromatography on silica gel column eluting with 0 to 3% gradient of ethyl acetate in petroleum ether to give compound 1-2. ¹H NMR (400 MHz, CDCl₃) δ 5.76 (ddd, J=17.2, 10.5, 6.5 Hz, 1 H), 5.14-5.03 (m, 2 H), 4.49 (d, J=3.2 Hz, 2 H), 4.12-4.03 (m, 1 H), 2.81-2.71 (m, 1 H), 2.36 (s, 1 H), 1.99-1.83 (m, 2 H), 1.55-1.49 (m, 3 H), 1.37 (d, J=0.7 Hz, 3 H).

### Step 3:

To a stirred solution of 7.3 g (39.67 mmol) of compound 1-2 and 31.3 g (396.0 mmol) of pyridine in 120 mL of DCM was added 16.8 g (59.55 mmol) trifluoromethanesulfonic anhydride dropwise at 0° C. The reaction mixture was stirred at 0° C. for 1 h and then quenched by addition of 20 mL of water at 0° C. It was extracted with three 60 mL portions of DCM. The combined organic extracts were washed with brine, and dried over anhydrous Na₂SO₄. After filtration, the filtrate was concentrated to give a residue, which was purified by chromatography on silica gel column eluting with 0 to 2% gradient of ethyl acetate in petroleum ether to afford compound 1-3. ¹H NMR (400 MHz, CDCl₃) δ 5.78 (ddd, J=17.1, 10.6, 6.2 Hz, 1 H), 5.21-5.07 (m, 2 H), 5.03 (dt, J=8.1, 5.4 Hz, 1 H), 4.65 (t, J=5.5 Hz, 1 H), 4.53 (dd, J=6.0, 2.0 Hz, 1 H), 2.95-2.85 (m, 1 H), 2.40 (dt, J=13.2, 7.6 Hz, 1 H), 2.15-2.04 (m, 1 H), 1.56 (s, 3H), 1.36 (s, 3 H).

### Step 4:

To a stirred solution of 10.0 g (65.1 mmol) of 4-chloro-7H-pyrrolo[2,3-d]pyrimidine in 120 mL THF was added 7.3 g (65.1 mmol) potassium tert-butoxide portionwise at room temperature. The reaction mixture was stirred at rt for 1 h and concentrated under vacuum. The residue was purified by trituration with 120 mL isopropyl ether. The solids were collected by filtration and washed with three 50 mL portions of isopropyl ether to give potassium 4-chloro-7H-pyrrolo[2,3-d]pyrimidin-7-ide salt.

To a stirred solution of 7.0 g (22.2 mmol) of the above potassium 4-chloro-7H-pyrrolo[2,3-d]pyrimidin-7-ide in 80 mL DMF was added a solution of 5.07 g (26.6 mmol) of compound 1-3 in 20 mL of DMF dropwise at 0° C. The reaction mixture was stirred at rt for 2 h and quenched by slow addition of water at 0° C. The mixture was extracted with three 50 mL portions of ethyl acetate. The combined organic extracts were washed with brine, and dried over Na₂SO₄. After filtration, the filtrate was concentrated to afford a residue, which was purified by chromatography on silica gel column eluting with 0 to 15% gradient of ethyl acetate in petroleum ether to afford compound 1-4. LC-MS: m/e=320 [M+H]⁺.

### Step 5:

A solution of 5.0 g (15.6 mmol) of compound 1-4 in 60 mL of NH₃.H₂O and 60 mL of THF in a sealed tube was stirred at 110° C. overnight. It was cooled to rt, diluted with 50 mL water, and extracted with three 80 mL portions of ethyl acetate. The combined organic extracts were washed with brine and dried over Na₂SO₄. After filtration, the filtrate was concentrated to afford a residue, which was purified by chromatography on silica gel column eluting with 0 to 75% gradient of ethyl acetate in petroleum ether to give compound 1-5. LC-MS: m/e=301 [M+H]⁺.

### Step 6:

To a stirred solution of 2.66 mL (1.33 mmol, 0.5 M in THF) of 9-borabicyclo[3.3.1]nonane was added 0.10 g (0.33 mmol) of compound 1-5. The reaction mixture was stirred at 50° C. for 1 h under N₂ atmosphere and cooled to room temperature. After addition of a solution of 0.35 g (1.66 mmol) of K₃PO₄ in 0.3 mL of H₂O, the mixture was stirred at rt for additional 30 min. To the mixture were added 0.09 g (0.30 mmol) of compound 16 and 0.024 g (0.03 mmol) of Pd(dppf)Cl₂. The reaction mixture was stirred at 50° C. for 1 h under N₂ atmosphere and quenched by addition of 20 mL of ice water. It was extracted with three 30 mL portions of ethyl acetate. The combined organic extracts were washed with brine, and dried over anhydrous Na₂SO₄. After filtration, the filtrate was concentrated to afford a residue, which was purified by chromatography on silica gel column eluting with 0 to 10% gradient of methanol in dichloromethane to afford compound 1-6. LC-MS: m/e=476 [M+H]⁺.

### Step 7:

To a stirred solution of 0.10 g (0.21 mmol) of compound 1-6 in 0.5 mL of methanol was added 3 mL of 4 N HCl in dioxane. The reaction mixture was stirred at rt for additional 1 h and diluted by addition of 8 mL of water. It was adjusted to pH 8 with saturated sodium bicarbonate and extracted with three 10 mL ports of DCM. The aqueous layer was concentrated to afford a residue, which was purified by Prep-HPLC [Column, XBridge Prep C18 OBD Column, 5 □m, 19*150 mm; Mobile phase, A: Water (10 mM NH₄HCO₃) and B: ACN (Gradient: 3% Phase B up to 28% in 10 min); Flow rate: 20 mL/min, t_{R} 9.42 min, Detector, 254 nm UV] to give compound 1-7. LC-MS: m/e=436 [M+H]⁺.

Using the procedures outlined in Method 1, steps 6-7, the following analogs in table 2 were made from compound 1-5 by employing the requisite aryl halide. Other compounds of Formula 1 may be prepared in a similar way.

TABLE 2
Synthesis of heterocyclic analogs

| | | LCMS |
|---|---|---|
| Aryl Halide | Example Structure | [M + 1]+ |
| 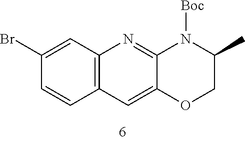 [SMILES: C[C@H]1COc2cc3ccc(Br)cc3nc2N1C(=O)OC(C)(C)C] | 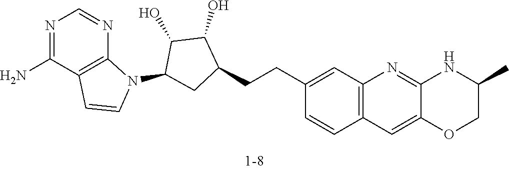 [SMILES: C[C@H]1COc2cc3ccc(CC[C@H]4C[C@@H](n5ccc6c(N)ncnc65)[C@H](O)[C@@H]4O)cc3nc2N1] | 461 |
| 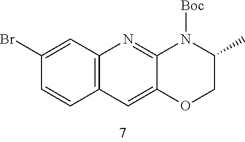 [SMILES: C[C@@H]1COc2cc3ccc(Br)cc3nc2N1C(=O)OC(C)(C)C] | 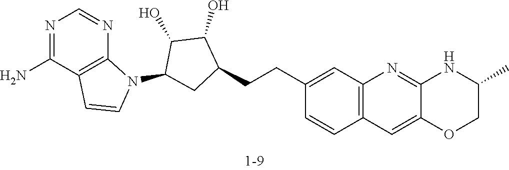 [SMILES: C[C@@H]1COc2cc3ccc(CC[C@H]4C[C@@H](n5ccc6c(N)ncnc65)[C@H](O)[C@@H]4O)cc3nc2N1] | 461 |
| 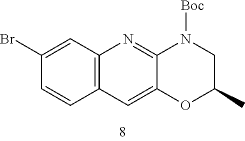 [SMILES: C[C@@H]1CN(C(=O)OC(C)(C)C)c2nc3cc(Br)ccc3cc2O1] | 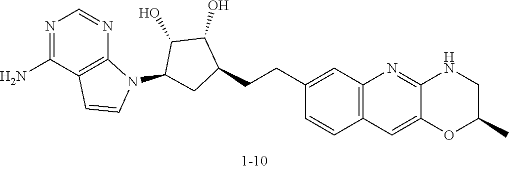 [SMILES: C[C@@H]1CNc2nc3cc(CC[C@H]4C[C@@H](n5ccc6c(N)ncnc65)[C@H](O)[C@@H]4O)ccc3cc2O1] | 461 |
| 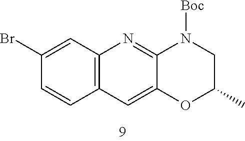 [SMILES: C[C@H]1CN(C(=O)OC(C)(C)C)c2nc3cc(Br)ccc3cc2O1] | 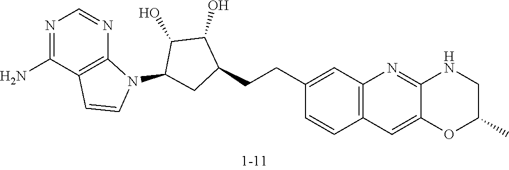 [SMILES: C[C@H]1CNc2nc3cc(CC[C@H]4C[C@@H](n5ccc6c(N)ncnc65)[C@H](O)[C@@H]4O)ccc3cc2O1] | 461 |
| 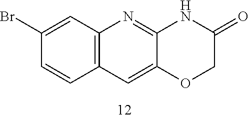 [SMILES: O=C1COc2cc3ccc(Br)cc3nc2N1] | 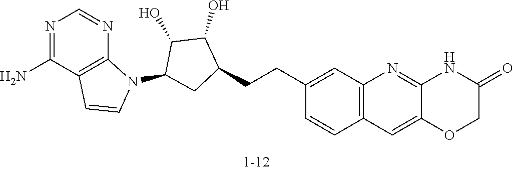 [SMILES: Nc1ncnc2c1ccn2[C@@H]1C[C@H](CCc2ccc3cc4c(nc3c2)NC(=O)CO4)[C@@H](O)[C@H]1O] | 461 |
| 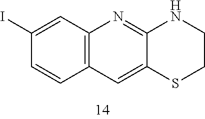 [SMILES: Ic1ccc2cc3c(nc2c1)NCCS3] | 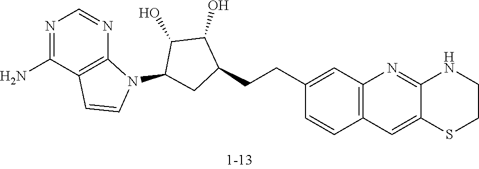 [SMILES: Nc1ncnc2c1ccn2[C@@H]1C[C@H](CCc2ccc3cc4c(nc3c2)NCCS4)[C@@H](O)[C@H]1O] | 463 |
| 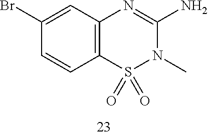 | 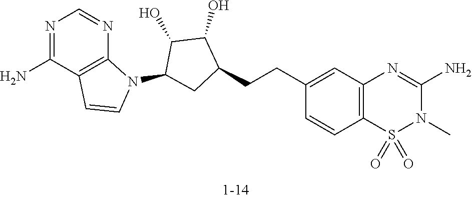 [SMILES: CN1C(N)=Nc2cc(CC[C@H]3C[C@@H](n4ccc5c(N)ncnc54)[C@H](O)[C@@H]3O)ccc2S1(=O)=O] | 472 |
| 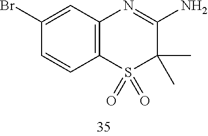 [SMILES: CC1(C)C(N)=Nc2cc(Br)ccc2S1(=O)=O] | 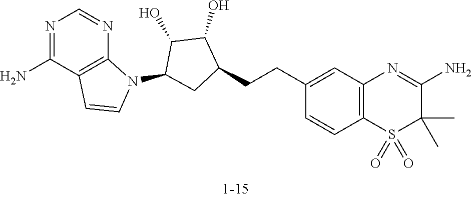 [SMILES: CC1(C)C(N)=Nc2cc(CC[C@H]3C[C@@H](n4ccc5c(N)ncnc54)[C@H](O)[C@@H]3O)ccc2S1(=O)=O] | 485 |
| 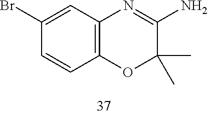 | 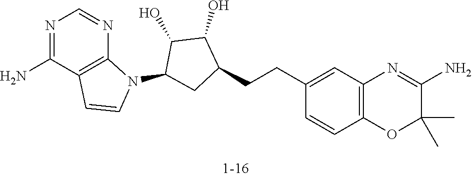 [SMILES: CC1(C)Oc2ccc(CC[C@H]3C[C@@H](n4ccc5c(N)ncnc54)[C@H](O)[C@@H]3O)cc2N=C1N] | 437 |
| 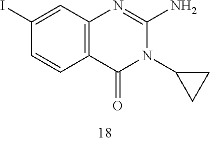 [SMILES: Nc1nc2cc(I)ccc2c(=O)n1C1CC1] | 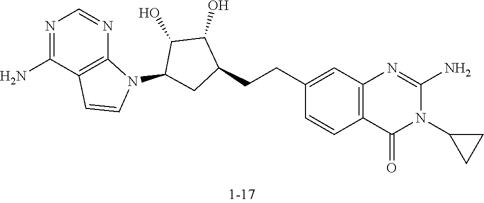 [SMILES: Nc1ncnc2c1ccn2[C@@H]1C[C@H](CCc2ccc3c(=O)n(C4CC4)c(N)nc3c2)[C@@H](O)[C@H]1O] | 462 |
| 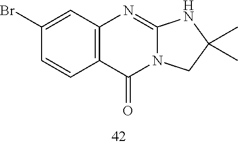 [SMILES: CC1(C)Cn2c(nc3cc(Br)ccc3c2=O)N1] | 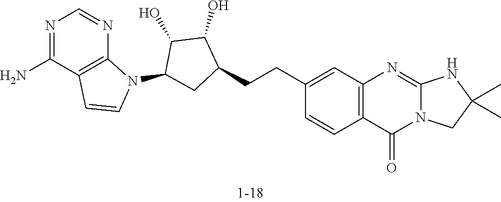 [SMILES: CC1(C)Cn2c(nc3cc(CC[C@H]4C[C@@H](n5ccc6c(N)ncnc65)[C@H](O)[C@@H]4O)ccc3c2=O)N1] | 476 |

### Method 2:

### EXAMPLE 2

### Synthesis of 3-amino-6-(2-((2R,3S,4R,5R)-5-(4-amino-7H-pyrrolo[2,3-d]pyrimidin-7-yl)-3,4-dihydroxytetrahydrofuran-2-yhethyl-2- methyl-2H-benzo[e][1,2,4]thiadiazine 1,1′-dioxide

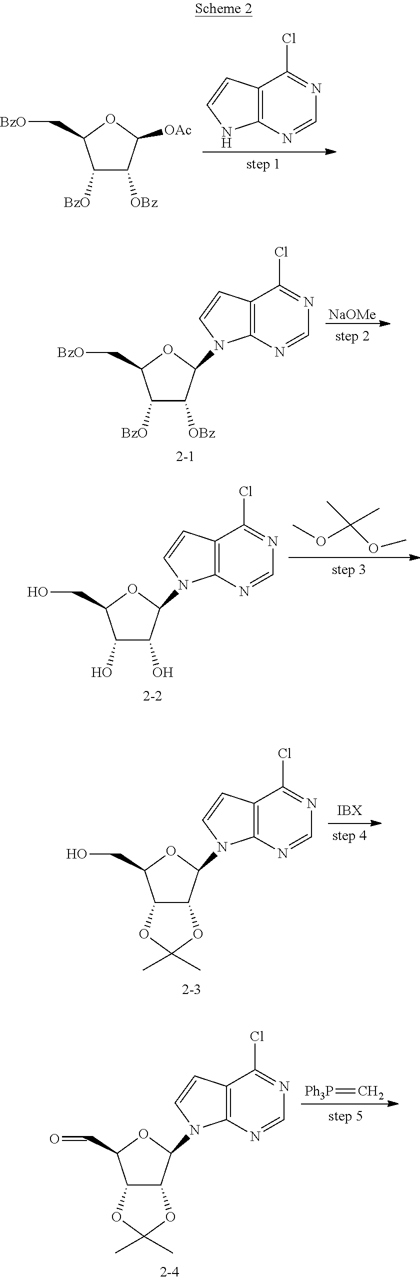

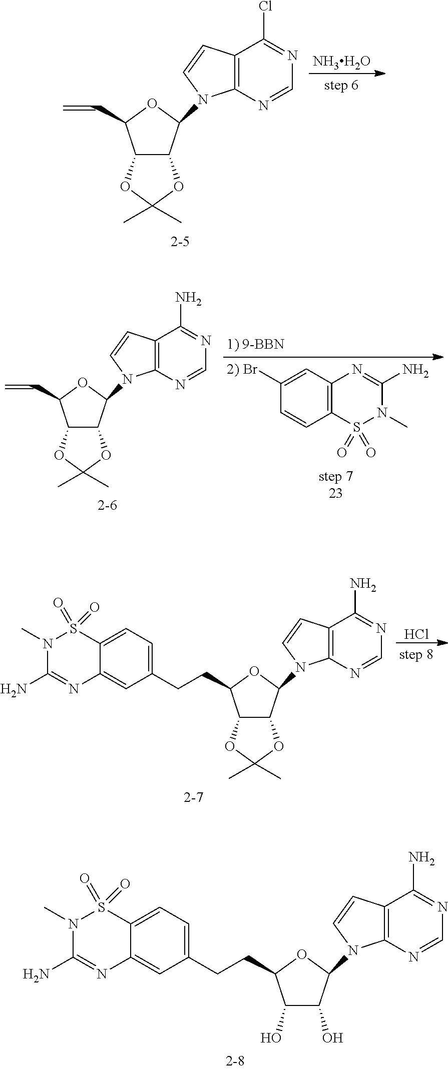

### Step 1:

To a solution of 10.0 g (65.4 mmol) of 4-chloro-7H-pyrrolo[2,3-d]pyrimidine in 250 mL of acetonitrile was added 16.0 g (78.5 mmol) of BSA. The resulting solution was stirred at rt for 40 min. After addition of 49.5 g (98.1 mmol) of (2S,3R,4R,5R)-2-(acetyloxy)-4-(benzoyloxy)-5-[(benzoyloxy)methyl]oxolan-3-yl benzoate and 22.0 g (98.1 mmol) of TMSOTf, the mixture was stirred at 85° C. for 2 h. The reaction was then quenched by addition of 500 mL of ice water and extracted with three 150 mL portions of ethyl acetate. The combined organic extracts were washed with 150 mL of brine, and dried over anhydrous sodium sulfate. After filtration, the filtrate was concentrated to give a residue, which was purified by chromatography on a silica gel column eluting with 0 to 5% gradient of ethyl acetate in petroleum ether to afford compound 2-1. LC-MS: m/e=598 [M+H]⁺.

### Step 2:

To a solution of 24.0 g (40.1 mmol) of compound 2-1 in 200 mL of methanol and 20 mL of dichloromethane was added 1.1 mg (0.02 mmol) of sodium methoxide. The solution was stirred at rt for 60 min; the solution was adjusted to pH 5-6 with 1N HCl solution. Then the mixture was concentrated; the solids were collected by filtration to afford compound 2-2. LC-MS: m/e=286 [M+H]⁺.

### Step 3:

To a solution of 10.0 g (35.0 mmol) of compound 2-2 in 200 mL of acetone was added 600 mg (3.48 mmol) of TsOH and 11.0 g (105.6 mmol) of 2,2-dimethoxypropane. The mixture was stirred at rt for 2 h. The reaction was then quenched by addition of 150 mL of water and extracted with three 150 mL portions of DCM. The combined organic extracts were washed with 150 mL of brine, and dried over anhydrous magnesium sulfate. After filtration, the filtrate was concentrated to afford compound 2-3, which was used in the next step without further purification. LC-MS: m/e=326 [M+H]⁺.

### Step 4:

To a solution of 10.0 g (30.7 mmol) of compound 2-3 in 110 mL of acetonitrile was added 12.9 g (46.1 mmol) of IBX. The mixture was stirred at 50° C. for 16 h and then cooled with an ice water bath. After filtration, the filtrate was concentrated to afford crude compound 2-4, which was used in the next step without further purification. LC-MS: m/e=324 [M+H]⁺.

### Step 5:

To a solution of 33.1 g (92.7 mmol) of bromo(methyl)triphenyl-lambda5-phosphane in 200 mL of THF was added 85 mL (85.0 mmol) of 1 M t-BuOK solution in THF. Then the mixture was stirred at 0° C. for 1 h, a solution of 10.0 g (30.9 mmol) of compound 2-4 in 10 mL of THF was introduced. The mixture was stirred at 0° C. for additional 1 h and then quenched by addition of 300 mL of saturated NH₄Cl solution. It was extracted with three 150 mL portions of ethyl acetate, the combined organic extracts were washed with 150 mL of brine, and dried over anhydrous sodium sulfate. After filtration, the filtrate was concentrated to afford a residue, which was purified by chromatography on a silica gel column eluting with 0 to 3% gradient of ethyl acetate in petroleum ether to afford 4.3 g of compound 2-5. LC-MS: m/e=322 [M+H]⁺.

### Step 6:

To a solution of 5.5 g (17.1 mmol) of compound 2-5 in 30 mL of 1,4-dioxane was added 30 mL of ammonia. The mixture was stirred at 100° C. for 20 h. Then the mixture was concentrated to afford 3.5 g of compound 2-6, which was used in the next step without further purification. LC-MS: m/e=303 [M+H]⁺.

### Step 7:

Compound 2-7 was prepared from compound 2-6, using the similar procedure described in Method 1, Step 6, by employing the intermediate 23 as the coupling partner. LC-MS: m/e=514 [M+H]⁺.

### Step 8:

Compound 2-8 was prepared from compound 2-7, using the similar procedure described in Method 1, Step 7. LC-MS (Shimadzu LC20AD/LCMS2020, Column:Shim-pack XR-ODS, 3.0*50 mm, 2.2 μm; Mobile Phase A:water/0.05% TFA,Mobile Phase B:ACN/0.05%, TFA; Flow rate: 1.2 mL/min; Gradient:5% B to 100% B in 2.0 min, hold 0.7 min; 190-400nm): m/e=474 [M+H]⁺.

Using the procedures outlined in Method 1, steps 6-7, the following analogs in table 3 were made from compound 2-6 by employing the requisite aryl halide. Other compounds of Formula 1 may be prepared in a similar way.

TABLE 3
Synthesis of heterocyclic analogs

| | | LCMS |
|---|---|---|
| Aryl Halide | Example Structure | [M + 1]+ |
| 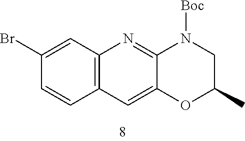 [SMILES: C[C@@H]1CN(C(=O)OC(C)(C)C)c2nc3cc(Br)ccc3cc2O1] | 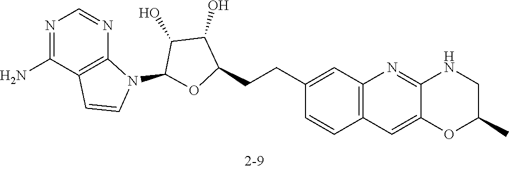 [SMILES: C[C@@H]1CNc2nc3cc(CC[C@H]4O[C@@H](n5ccc6c(N)ncnc65)[C@H](O)[C@@H]4O)ccc3cc2O1] | 463 |
| 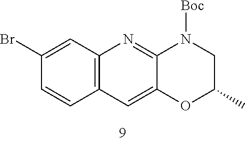 [SMILES: C[C@H]1CN(C(=O)OC(C)(C)C)c2nc3cc(Br)ccc3cc2O1] | 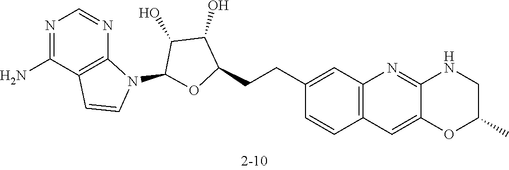 [SMILES: C[C@H]1CNc2nc3cc(CC[C@H]4O[C@@H](n5ccc6c(N)ncnc65)[C@H](O)[C@@H]4O)ccc3cc2O1] | 463 |
| 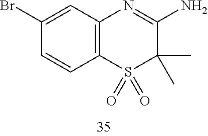 [SMILES: CC1(C)C(N)=Nc2cc(Br)ccc2S1(=O)=O] | 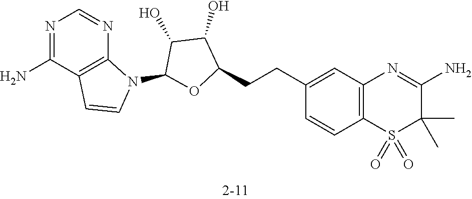 [SMILES: CC1(C)C(N)=Nc2cc(CC[C@H]3O[C@@H](n4ccc5c(N)ncnc54)[C@H](O)[C@@H]3O)ccc2S1(=O)=O] | 487 |
| 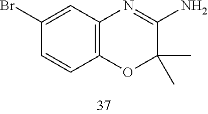 | 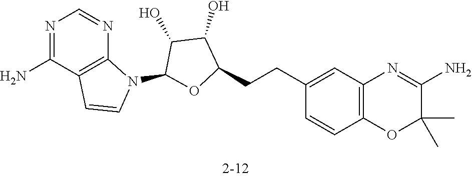 [SMILES: CC1(C)Oc2ccc(CC[C@H]3O[C@@H](n4ccc5c(N)ncnc54)[C@H](O)[C@@H]3O)cc2N=C1N] | 439 |

### Method 3:

### EXAMPLE 3

### Synthesis of N-(4-chlorophenyl)-6-(6-fluoroquinolin-4-yl)-6-azaspiro[2.5]octane-1-carboxamide

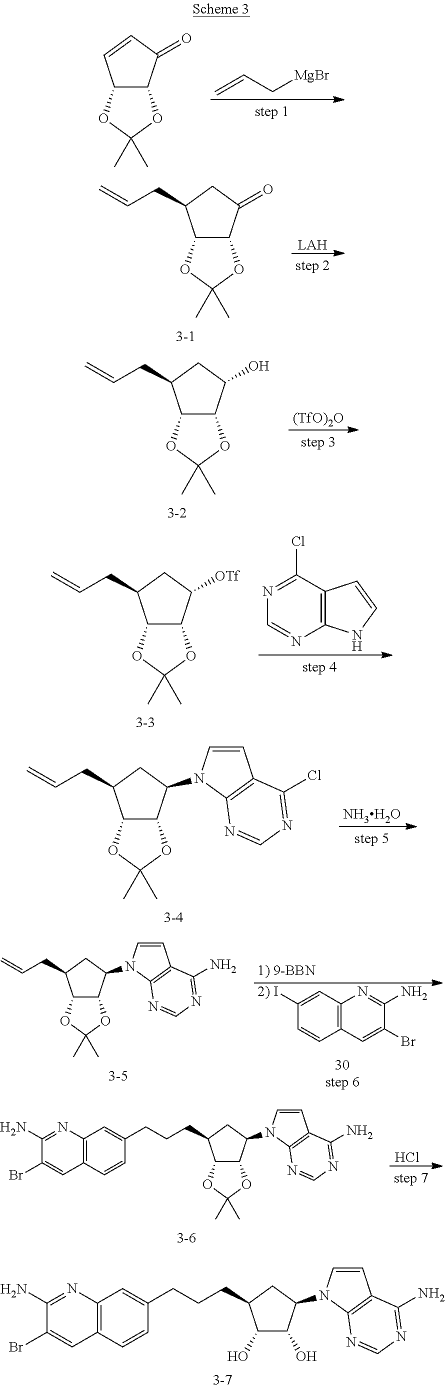

### Step 1:

A solution of 9.2 g (48.7 mmol) of Cul and 2.4 g (73 mmol) of LiCI in 50 mL of THF was stirred at room temperature for 5 min. and cooled to −78° C. After addition of 58 mL (1M in THF, 73 mmol) of bromo (prop-2-en-1-yl) magnesium dropwise, the mixture was stirred at −78° C. for 30 min under nitrogen atmosphere. To the mixture were added 6.9 mL (38.0 mmol) of chlorotrimethylsilane, 9.9 mL (48.7 mmol) of HMPA and a solution of 3.0 g (19.0 mmol) of (3aR,6aR)-5,5-dimethyl-1,3a,4,5,6,6a-hexahydropentalen-1-one in 10 mL of THF. The resulting mixture was then stirred at room temperature for 2 h and quenched with 20 mL of NH₄Cl at 0° C. The mixture was extracted with three 10 mL portions of ethyl acetate; the combined organic extracts were washed with brine. It was dried over anhydrous Na₂SO₄, and filtered; the filtrate was concentrated under reduced pressure to give a residue, which was purified by silica gel column chromatography eluting with 0% to 19% ethyl acetate in petroleum ether to afford compound 3-1.

### Step 2:

To a stirred solution of 0.35 g (9.1 mmol) of LAH in 10 mL of THF was added a solution of 1.2 g (6.0 mmol) of compound 3-1 in THF dropwise at 0° C. under nitrogen atmosphere. The mixture was stirred at room temperature for 2 h and quenched by the addition of 0.35 mL of water, 0.35 mL of 15% NaOH and 1.05 mL of water at 0° C. The resulting mixture was filtered; the filter cake was washed with ethyl acetate. The filtrate was concentrated under reduced vacuum to give a residue, which was purified by silica gel column chromatography eluting with 0% to 3% ethyl acetate in petroleum ether to afford compound 3-2. ¹H NMR (400 MHz, DMSO-d₆) δ 5.75 (ddt, J=17.0, 10.3, 6.6 Hz, 1 H), 5.11-4.95 (m, 2 H), 4.3-4.30 (m, 2 H), 4.19 (d, J=5.7 Hz, 1 H), 4.09-3.88 (m, 1 H), 2.09-1.71 (m, 4 H), 1.56-1.43 (m, 1 H), 1.38 (s, 3 H), 1.23 (s, 3 H).

### Step 3:

To a stirred solution of 0.70 g (3.53 mmol) of compound 3-2 and 2.8 g (35.4 mmol) of pyridine in 20 mL of dichloromethane was added a solution of 1.5 g (5.32 mmol) of trifluoromethane sulfonyl trifluoromethanesulfonate in 2 mL of dichloromethane at 0° C. dropwise. The reaction mixture was stirred at 0° C. for 2 h and quenched by addition of 10 mL of water at 0° C. The resulting mixture was extracted with three 10 mL portions of dichloromethane. The combined organic extracts were washed with 10 mL of brine, and dried over anhydrous Na₂SO₄. After filtration, the filtrate was concentrated under reduced pressure to afford a crude, which was purified by chromatography on silica gel column eluting with 0% to 100% ethyl acetate in petroleum ether to give compound 3-3.

### Step 4:

To a stirred solution of 0.59 g (3.34 mmol) of potassium 4-chloro-7H-pyrrolo[2,3-d]pyrimidin-7-ide in 10 mL of DMF was added 0.85 g (2.57 mmol) of compound 3-3 dropwise at 0° C. The reaction mixture was stirred at room temperature for 2 h and quenched by the addition of 10 mL of water at 0° C. The mixture was extracted with three 10 mL portion of ethyl acetate. The combined organic extracts were washed with 10 mL of brine, and dried over anhydrous Na₂SO₄. It was filtered and the filtrate was concentrated. The residue was purified by silica gel column chromatography eluting with 0% to 30% ethyl acetate in petroleum ether to afford compound 3-4. LC-MS: m/e=334 [M+H]⁺.

### Step 5:

To a stirred solution of 0.20 g (0.60 mmol) of compound 3-4 7 in 5 mL of THF was added 5 mL of amine hydrate. The mixture in a sealed tube was stirred at 110° C. overnight and cooled to rt. The mixture was extracted with three 10 mL portions of ethyl acetate; the combined organic extracts were washed with 10 mL of brine, and dried over anhydrous Na₂SO₄. After filtration, the filtrate was concentrated under reduced pressure to afford a crude, which was purified by chromatography on silica gel column eluting with 0% to 80% ethyl acetate in petroleum ether to afford compound 3-5. LCMS: m/e=315 [M+H]⁺.

### Step 6:

Using the procedure described in Method 1, Step 6, compound 3-5 was converted to compound 3-6, employing intermediate 30 as the coupling partner. LC-MS: m/e=537, 539 [M+H]⁺.

### Step 7:

Using the procedure described in Method 1, Step 7, compound 3-6 was converted to compound 3-7 similarly. LC-MS (conditions: Shimadzu LC2OADXR/LCMS2020, Column: CORTECS C18 100A (2.1*50 mm), 2.7 μm; Mobile phase A: 0.1% FA in Water, B:Acetonitrile; Gradient: 90:10 to 0:100 (A:B) over 2.0 min, 0:100 (A:B) for 0.60 min, Flow rate: 1.0 mL/min; UV detection: 190-400 nm): m/e=497, 499 [M+H]⁺.

### Method 4:

### EXAMPLE 4

### Synthesis of 7-(2-((1S,2R,3S,4R)-4-(4-amino-7H-pyrrolo[2,3-dpyrimidin-7-yl]-2,3-dihydroxycyclopentyl)ethyl-2H-[1,4]thiazino[3,2-b]quinoline-3(4H)-one

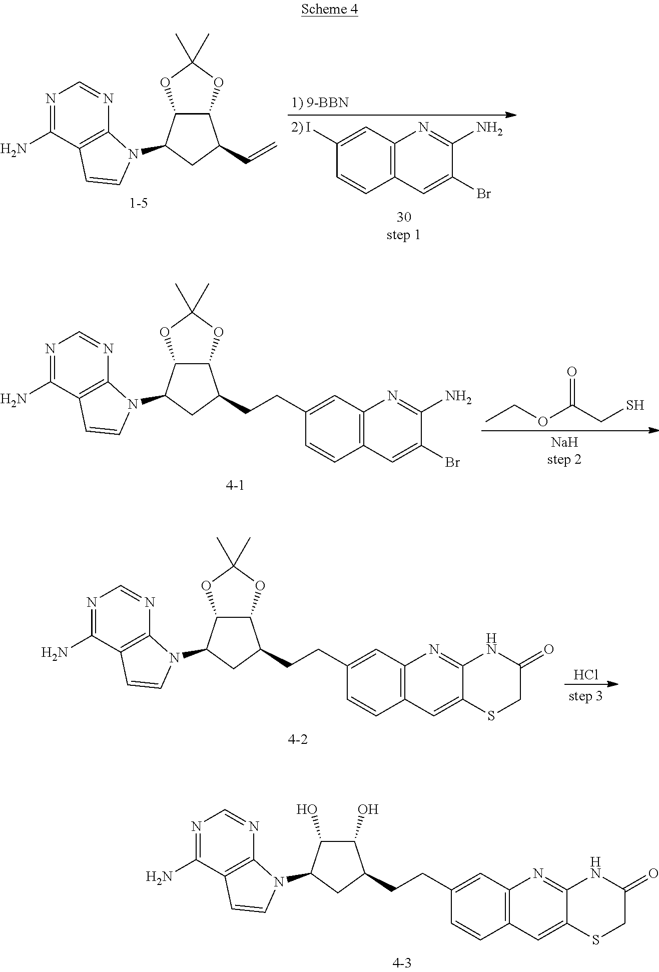

### Step 1:

Using the procedure described in Method 1, Step 6, compound 4-1 was prepared from compound 1-5 by employing intermediate 30 as the coupling partner. LC-MS: m/e=523, 525 [M+H]⁺.

### Step 2:

Using the procedure described in Method 6, Step 1, compound 4-2 was prepared from compound 4-1 similarly. LC-MS: m/e=517 [M+H]⁺.

### Step 3:

Using the procedure described in Method 1, Step 7, compound 4-3 was prepared from compound 4-2 similarly. LC-MS (Shimadzu LC2OADXR/LCMS2020, Column: KinetexEVO C18(50*3.0 mm) 2.6 μm; Mobile Phase A: 0.04% Ammonium Bicarbonate in water, B: Acetonitrile; Gradient: 90:10 to 5:95 (A:B) in 2.1 min, 5:95 (A:B) hold for 0.6 min; Flow rate: 1.2 mL/min; UV detection: 190-400 nm): m/e=477 [M+H]⁺.

### Synthesis of Intermediates

1. Synthesis of intermediate 6:

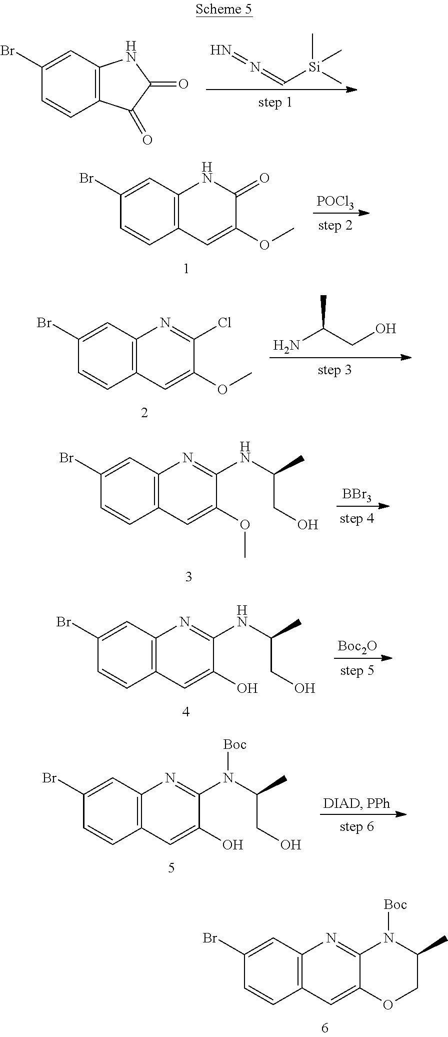

### Step 1:

To a stirred solution of 5.0 g (22.1 mmol) of 6-bromo-2, 3-dihydro-1H-indole-2, 3-dione and 22 mL (44.2 mmol) of (diazomethyl) trimethylsilane hexane was added 4.5 g (44.2 mmol) of triethylamine in 30 mL of ethanol. The mixture was stirred for 18 h at room temperature under N₂ atmosphere. It was filtered and the filter cake was washed with 30 mL of ethyl acetate to afford compound 1. LC-MS: m/e=254, 256 [M+H]⁺.

### Step 2:

To a stirred solution of 3.68 g (14.5 mmol) of compound 1 in 30 mL of toluene was added 14.5 g (94.5 mmol) of phosphoroyl trichloride dropwise at room temperature. The resulting mixture was stirred at 100° C. for 1 h, cooled down to room temperature, and quenched with ice-water. The mixture was basified to pH 7 with NaOH and extracted with three 15 mL portions of DCM. The combined organic layers were washed with brine, and dried over anhydrous Na₂SO₄. After filtration, the filtrate was concentrated under reduced pressure to afford a residue, which was purified by chromatography on silica gel column eluting with 25% ethyl acetate in petroleum ether to afford 2.3 g compound 2. LC-MS: m/e=272, 274 [M+H]⁺.

### Step 3:

To a stirred solution of 0.50 g (1.83 mmol) of compound 2 in 2.5 mL of pyridine was added 2.5 mL of (2S)-2-aminopropan-1-ol at room temperature. The reaction mixture was irradiated with microwave radiation at 150° C. for 1.5 h. The mixture was cooled down to room temperature and extracted with three 15 mL portions of ethyl acetate. The combined organic layers were washed with brine, and dried over anhydrous Na₂SO₄. After filtration, the filtrate was concentrated under reduced pressure to afford a residue, which was purified by chromatography on silica gel column eluting with 25% ethyl acetate in petroleum ether to afford 550 mg compound 3. LC-MS: m/e=311, 313 [M+H]⁺.

### Step 4:

To a stirred solution of 0.56 g (1.78 mmol) of compound 3 in 5 mL of DCM were added 1.34 g (5.35 mmol) of tribromoborane at room temperature. The reaction mixture was stirred at room temperature for 2 h and concentrated under reduced pressure to afford a residue, which was triturated with 30 mL of petroleum ether to get compound 4. LC-MS: m/e=297, 299 [M+H]⁺.

### Step 5:

To a stirred solution of 0.48 g (1.62 mmol) of compound 4 in 12 mL of DCM was added 0.20 g (1.95 mmol) of triethylamine and 0.35 g (1.62 mmol) of di-tert-butyldiarbonate at room temperature. The reaction mixture was stirred at room temperature for 3 h. The mixture was diluted with 20 mL of DCM, then quenched with 0.060 g (0.81 mmol) of di-ethylamine and washed with three 20 mL portions of NH₄Cl aqueous. The organic layer was dried over anhydrous Na₂SO₄ and filtered; the filtrate was concentrated to afford a residue, which was purified by chromatography on silica gel column eluting with 25% ethyl acetate in petroleum ether to get compound 5. LC-MS: m/e=397, 399 [M+H]⁺.

### Step 6:

To a stirred solution of 0.35 g (0.89 mmol) of compound 5 in 8 mL of THF was added 0.28 g (1.07 mmol) of PPh₃ and 022 g (1.07 mmol) of DIAD dropwise at room temperature. The mixture was stirred at room temperature overnight, and concentrated under reduced pressure to afford a residue, which was purified by chromatography on silica gel column eluting with 25% ethyl acetate in petroleum ether to afford compound 6. LC-MS: m/e=379, 381 [M+H]⁺.

Using the procedures outlined in Method 3, steps 3-6, the following intermediates in Table 4 were made from compound 2 by employing the requisite aminoalcohol. Other intermediates described herein may be prepared in a similar way.

TABLE 4
Synthesis of intermediates

| Intermediate Structure | LCMS [M + 1]+ |
|---|---|
| 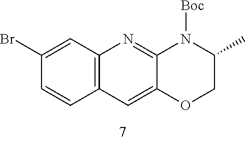 [SMILES: C[C@@H]1COc2cc3ccc(Br)cc3nc2N1C(=O)OC(C)(C)C] | 379, 381 |
| 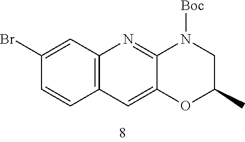 [SMILES: C[C@@H]1CN(C(=O)OC(C)(C)C)c2nc3cc(Br)ccc3cc2O1] | 379, 381 |
| 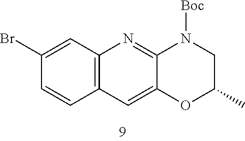 [SMILES: C[C@H]1CN(C(=O)OC(C)(C)C)c2nc3cc(Br)ccc3cc2O1] | 379, 381 |

### 2. Synthesis of Intermediate 12:

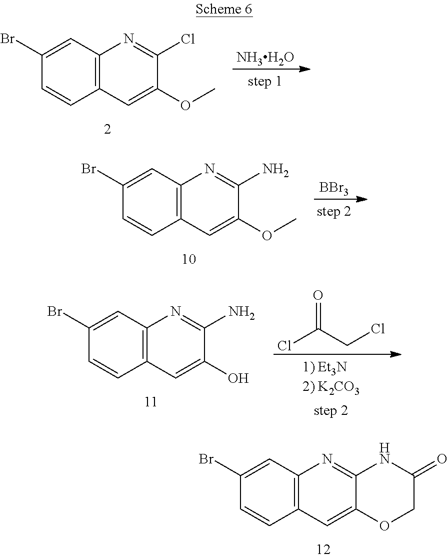

### Step 1:

To a stirred solution of 0.50 g (1.84 mmol) of compound 2 in 9 mL of dioxane was added 9 mL of NH₃.H₂O. The reaction mixture in a sealed tube was stirred at 120° C. overnight and cooled to rt. The mixture was extracted with three 15 mL portions of ethyl acetate; the combined organic extracts were washed with brine, and dried over anhydrous Na₂SO₄. After filtration, the filtrate was concentrated to afford a residue, which was purified by chromatography on silica gel column eluting with 0 to 30% gradient of ethyl acetate in petroleum ether to afford compound 10. LC-MS: m/e=253, 255 [M+H]⁺.

### Step 2:

Using the procedure described in Method 4, Step 4, compound 10 was converted to compound 11. LCMS: m/e=239, 241 [M+H]⁺.

### Step 3:

To a stirred solution of 0.16 g (0.67 mmol) of compound 11 in 8 mL THF was added 0.20 g (2.02 mmol) of Et₃N and 0.10 g (0.87 mmol) of 2-chloroacetyl chloride dropwise at 0° C. The reaction mixture was stirred at room temperature for 2 h. after addition of 0.19 g (1.34 mmol) of K₂CO₃, the mixture was stirred at 50° C. for 1 h and cooled to rt. The mixture was extracted with three 15 mL portions of ethyl acetate; the combined organic extracts were washed with brine, and dried over anhydrous Na₂SO₄. After filtration, the filtrate was concentrated to afford a residue, which was purified by Prep-TLC with ethyl acetate/petroleum ether (1:3) to afford compound 12. LC-MS: m/e=279, 281 [m+H]⁺.

### 3. Synthesis of Intermediate 14:

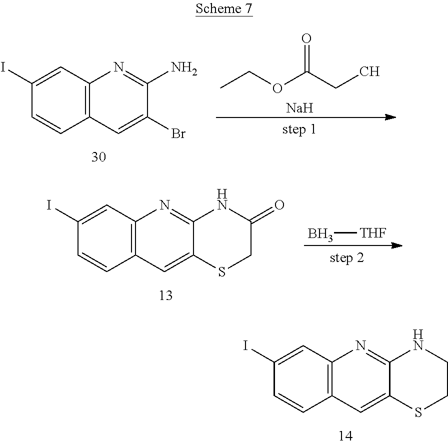

### Step 1:

To a stirred solution of 0.62 g (5.16 mmol) of ethyl 2-mercaptoacetate in 4 mL of DMF was added 0.12 g (5.16 mmol) of NaH in portion at 0° C. The solution was stirred at RT for 1 h, 0.60 g (1.72 mmol) of compound 30 was added. The mixture was stirred at 50° C. for 3 h, cooled to 0° C., and quenched by addition 5 mL of water. The mixture was extracted with three 15 mL portions of ethyl acetate. The combined organic extracts were washed with brine, and dried over anhydrous Na₂SO₄. After filtration, the filtrate was concentrated to afford a residue, which was purified by C18 silica gel, eluted with 0 to 50% gradient of acetonitrile in water (0.1% NH₄HCO₃) to afford compound 13. LC-MS: m/e=343 [M+H]⁺.

### Step 2:

To a stirred solution of 0.08 g (0.234 mmol) of compound 13 in 1 mL of THF was added 4.6 mL (4.68 mmol, 1 M) of BH₃-THF dropwise. The mixture was stirred at 70° C. for 3 h under nitrogen atmosphere, cooled to rt and quenched by addition of 1 mL of HCl (1 N in THF) at 0° C. It was then stirred at 60° C. for addiction 0.5 h, and cooled to room temperature. It was adjusted to pH 9 with saturated NaHCO₃ solution, and extracted with three 10 mL portions of ethyl acetate. The combined organic layers were washed with brine, and dried over anhydrous Na₂SO₄. After filtration, the filtrate was concentrated under vacuum to afford a residue, which was purified by Prep-TLC with 0 to 30% gradient of ethyl acetate in petroleum ether to afford compound 14. LC-MS: m/e=329 [M+H]⁺.

### 4. Synthesis of Intermediate 16:

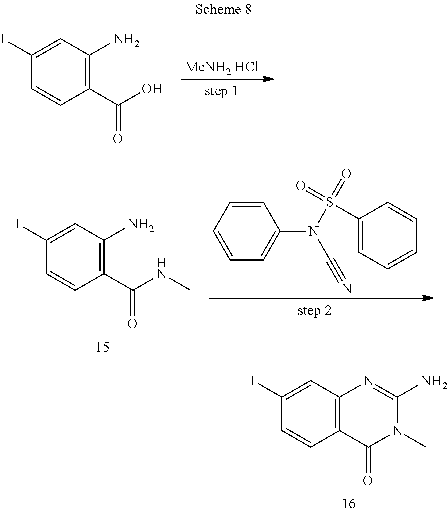 [SMILES: "MeNH2 HCl" ; Nc1cc(I)ccc1C(=O)O ; CNC(=O)c1ccc(I)cc1N ; N#CN(c1ccccc1)S(=O)(=O)c1ccccc1 ; Cn1c(N)nc2cc(I)ccc2c1=O]

### Step 1:

To a stirred solution of 1.0 g (3.80 mmol) 2-amino-4-iodobenzoic acid, 0.31 g (4.56 mmol) methylamine hydrochloride and 1.47 g (11.41 mmol) of N, N-Diisopropylethylamine in 12 mL of DCM were added 0.87 g (4.56 mmol) of EDCI and 0.62 g (4.56 mmol) of HOBT at rt. The mixture was stirred for additional 2 h at rt and quenched by addition of 10 mL of water. It was extracted with two 30 mL portions of DCM; the combined organic extracts were washed with brine, and dried over anhydrous Na₂SO₄. After filtration, the filtrate was concentrated to afford a residue, which was purified by chromatography on silica gel column eluted with 0 to 65% gradient of ethyl acetate in petroleum ether to afford compound 15. LC-MS: m/e=277 [M+H]⁺.

### Step 2:

To a stirred solution of 0.30 g (1.09 mmol) of compound 15 in 8 mL of dioxane was added 0.28 g (1.09 mmol) of N-cyano-N-phenylbenzenesulfonamide and 3.24 mL (3.24 mmol, 1 M in THF) of LiHMDS at rt. The resulting mixture was stirred at 100° C. for 1 h and cooled to rt. The reaction was quenched by addition of 25 mL of water and extracted with three 20 mL portions of ethyl acetate. The combined organic extracts were washed with brine, and dried over anhydrous Na₂SO₄. After filtration, the filtrate was concentrated to afford a residue, which was purified by chromatography on silica gel column eluted with 0 to 40% gradient of ethyl acetate in petroleum ether to afford compound 16. LC-MS: m/e=302 [M+H]⁺.

### 5. Synthesis of Intermediate 18:

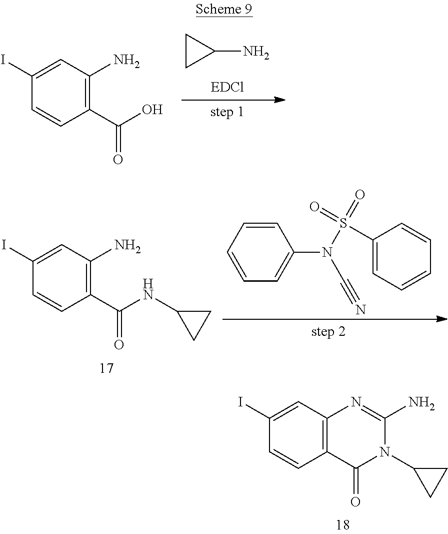

### Step 1:

Following the procedures described in Scheme 8, step 1, compound 17 was prepared similarly. LC-MS: m/e=303 [M+H]⁺.

### Step 2:

Following the procedures described in Scheme 8, step 2, compound 18 was prepared from 17 similarly. LC-MS: m/e=328 [M+H]⁺.

### 6. Synthesis of Intermediate 23:

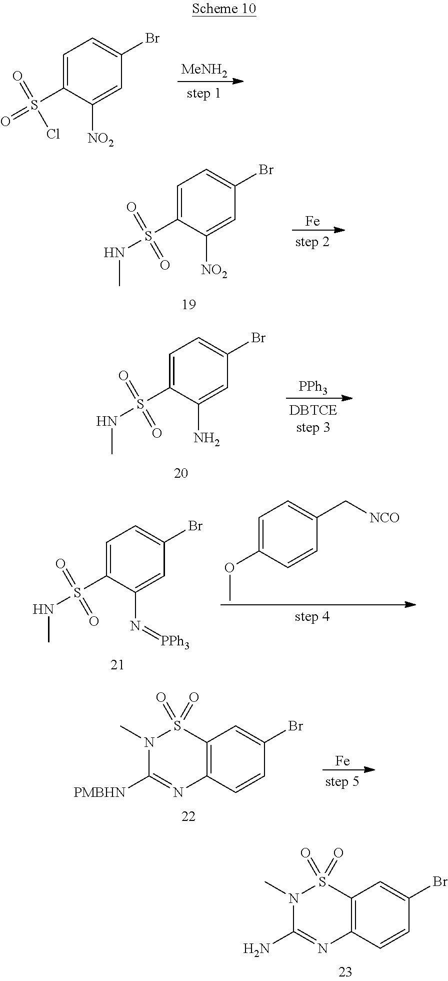

### Step 1:

To a solution of 2.8 g (9.3 mmol) of 4-bromo-2-nitrobenzene-1-sulfonyl chloride in 10 mL of MeOH was added 10 mL (1 N, 10 mmol) of methylamine in THF. The mixture was stirred at 50° C. for 1 h and concentrated; the residue was purified by Prep-HPLC (Column, C18 silica gel; mobile phase, ACN/H₂O=5/5 increasing to ACN/H₂O=95/5; Detector) to afford compound 19. ¹H NMR (300 MHz, DMSO-d₆) δ 8.38 (d, J=1.9 Hz, 1 H), 8.12 (dd, J=8.5, 2.0 Hz, 1 H), 8.02 (s, 1H), 7.88 (d, J=8.5 Hz, 1 H), 2.55 (s, 3 H).

### Step 2:

To a solution of 1.5 g (5.76 mmol) of compound 19 in 30 mL of MeOH was added 1.2 g (20 mmol) of iron powder and 20 mL of conc. HCl solution. The mixture was stirred for 1 h at room temperature. After filtration, the filtrate was concentrated to give a residue, which was purified with chromatography on a silica gel column with 50% gradient of ethyl acetate in petroleum ether to afford compound 20. LC-MS: m/e=265, 267 [M+H]⁺.

### Step 3:

To a solution of 0.60 g (2.26 mmol) of compound 20 in 20 mL of DCM was added 0.89 g of PPh₃ (3.39 mmol), 0.92 g of Et₃N (9.05 mmol) and 1.1 g (3.39 mmol) of DBTCE. The mixture was stirred at room temperature overnight. The mixture was concentrated; the residue was purified by Prep-HPLC (Column, silica gel; mobile phase, ACN/H₂O=5/5 increasing to ACN/H₂O=95/5; Detector) to give compound 21. LC-MS: m/e=525, 527 [M+H]⁺.

### Step 4:

To a solution of 0.40 g (0.76 mmol) of compound 21 in 10 mL of o-xylene was added 0.24 g (1.5 mmol) of 1-(isocyanoatomethyl)-4-methoxybenzene. The reaction mixture was irradiated in microwave at 140° C. for 30 min. The mixture was concentrated to afford crude compound 22, which was used in the next step without further purification. LC-MS: m/e=410, 412 [M+H]⁺.

### Step 5:

A solution of 0.45 g of crude compound 22 in 10 mL of TFA was irradiated in microwave at 160° C. for 30 min. The cooled mixture was diluted with 20 mL of water and adjusted to pH 10 with 1N NaHCO₃ solution. The solution was extracted with three 30 mL portions ethyl acetate; the combined organic extracts were concentrated. The residue was purified by Prep-HPLC (Column, silica gel; mobile phase, ACN/H₂O=5/5 increasing to ACN/H₂O=95/5; Detector) to give compound 23. LC-MS: m/e=290, 292 [M+H]⁺.

### 7. Synthesis of Intermediate 30:

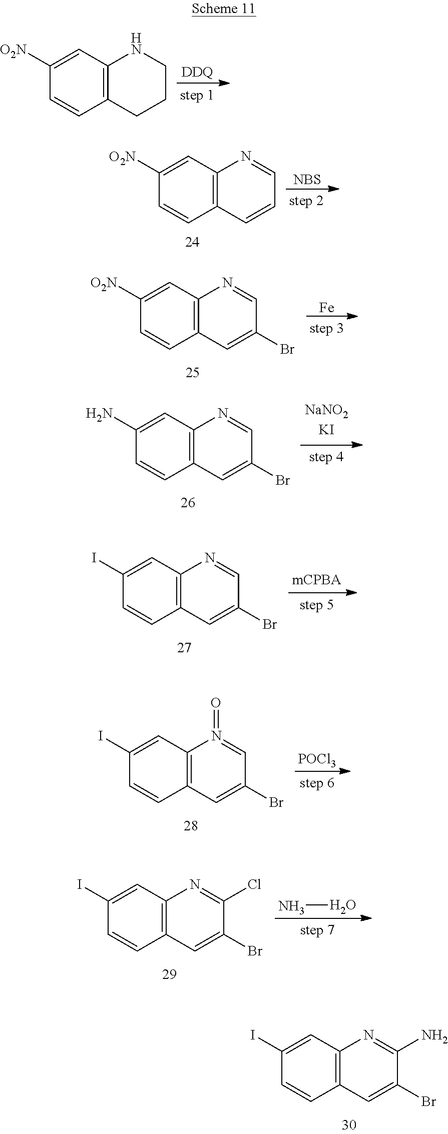

### Step 1:

To a stirred solution of 20.0 g (112 mmol) of 7-nitro-1,2,3,4-tetrahydroquinoline in 600 mL of dichloromethane was added 50.8 g (224 mmol) of 2,3-dichloro-5,6-dicyano-1,4-benzoquinone in several batches at 0° C. The mixture was stirred at room temperature for 3 h and filtered; the filter cake was washed with three 200 mL portions of dichloromethane. The filtrate was concentrated under vacuum to afford compound 24. LC-MS: m/e=175 [M+H]⁺.

### Step 2:

To a stirred solution of 23.0 g (132 mmol) of compound 24 in 180 mL of acetic acid was added 30.2 g (170 mmol) of NBS in several batches at room temperature. The reaction mixture was stirred at 110° C. for 2 h and cooled to rt. The mixture was filtered; the filter cake was washed with three 150 mL portions of tert-butyl methyl ether to compound 25. LC-MS: m/e=253, 255 [M+H]⁺.

### Step 3:

To a stirred solution of 18.5 g (73.4 mmol) of compound 25 in 120 mL of ethanol and 80 mL of H₂O were added 15.7 g (293.6 mmol) of NH₄Cl and 20.5 g (367 mmol) of iron powder. The mixture was stirred at 80° C. for 2 h under nitrogen atmosphere and cooled to rt. The mixture was filtered; the filter cake was washed with three 150 mL portions of dichloromethane. The filtrate was extracted with three 300 mL portions of dichloromethane. The combined organic extracts were washed with brine, and dried over anhydrous Na₂SO₄. After filtration, the filtrate was concentrated under vacuum to afford compound 26. LC-MS: m/e=223, 225 [M+H]⁺.

### Step 4:

To a stirred 125 mL of ice water was added 100 mL of conc. H2SO₄ dropwise at 0° C. Then 10.0 g (45.0 mmol) of compound 26 was added in several batches at 0° C. After 10 min, a solution of 6.2 g (90 mmol) of NaNO₂ in 10 mL of H₂O was added dropwise at 0° C. After 20 min, a solution of 20.2 g (135 mmol) of Nal in 10 mL of H₂O was added dropwise. The mixture was stirred for additional 30 min at 0° C., then warmed to 60° C. and stirred for 2 h. The mixture was diluted with 150 mL of water, adjusted to pH 8-9 with 2 N NaOH and extracted with three 200 mL of ethyl acetate. The combined organic extracts were washed with brine, and dried over anhydrous Na₂SO₄. After filtration, the filtrate was concentrated under vacuum to afford a crude, which was purified by chromatography on silica gel column eluting with 0 to 1% gradient of ethyl acetate in petroleum ether to afford compound 27. LC-MS: m/e=334, 336 [M+H]⁺.

### Step 5:

To a stirred solution of 5.2 g (15.6 mmol) of compound 27 in 80 mL of dichloromethane was added 8.05 g (46.8 mmol) of m-CPBA in several batches at 0° C. The reaction mixture was stirred at room temperature overnight and filtered; the filtrate was diluted with 100 mL of water. adjusted to pH 7-8 with saturated NaHCO₃ solution. It was extracted with three 80 mL portions of dichloromethane; the combined organic extracts were washed with brine, and dried over Na₂SO₄. After filtration, the filtrate was concentrated to afford a crude, which was purified by chromatography on silica gel column eluting with 0 to 15% gradient of ethyl acetate in petroleum ether to afford compound 28. LC-MS: m/e=350, 352 [M+H]⁺.

### Step 6:

To a stirred solution of 2.5 g (7.14 mmol) of compound 28 in 60 mL of chloroform was added 7.7 g (50.22 mmol) of POCl₃dropwise. The mixture was stirred at 80° C. for 2 h and cooled down to room temperature. The reaction was quenched by addition of 80 mL of water at 0° C. It was adjusted pH to 7-8 with saturated NaHCO₃ solution, and extracted with three 80 mL portions of dichloromethane. The combined organic extracts were washed with brine, and dried over anhydrous Na₂SO₄. After filtration, the filtrate was concentrated under vacuum to afford a crude, which was purified by chromatography on silica gel column eluting with 0 to 3% gradient of ethyl acetate in petroleum ether to afford compound 29. LC-MS: m/e=368, 370 [M+H]⁺.

### Step 7:

To a stirred solution of 0.20 g (1.63 mmol) of compound 29 in 6 mL of 1,4-dioxane was added 4 mL of ammonium hydroxide. The resulting solution in a sealed tube was stirred at 120° C. overnight and cooled to rt. The mixture was extracted with three 10 mL portions of ethyl acetate; the combined organic extracts were washed with 10 mL of brine and dried over anhydrous Na₂SO₄. After filtration, the filtrate was concentrated under vacuum to afford a crude, which was purified by chromatography on silica eluted with eluting with 0% to 20% ethyl acetate in petroleum ether to afford compound 30. LC-MS: m/e=349, 351 [M+H]⁺.

### 8. Synthesis of Intermediate 35:

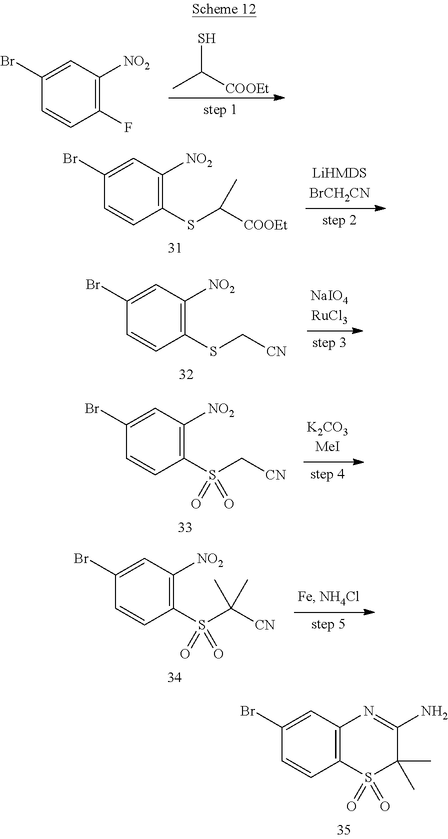

### Step 1:

To a solution of 21 g (9.09 mmol) of 4-bromo-1-fluoro-2-nitrobenzene in 30 mL of DMF was added 3.1 g (22.7 mmol) of K₂CO₃ and 1.2 g (9.1 mmol) of ethyl 2-sulfanylpropanoate. The solution was stirred at 30° C. for 4 h. The reaction was then quenched by addition of 100 mL of water and extracted with three 50 mL portions of ethyl acetate; the organic extracts were dried over anhydrous sodium sulfate and concentrated. The residue was chromatographed on a silica gel column eluting with ethyl acetate/petroleum ether (1/4) to compound 31.

### Step 2:

Into a 50-mL 3-necked round-bottom flask purged and maintained with an inert atmosphere of argon were placed 100 mg (0.30 mmol) of compound 31 and 5 mL of oxolane. To this solution was added 60 mg (0.36 mmol) of LiHMDS dropwise with stirring. The solution was stirred at −78° C. for 1 h, a solution of 72 mg (0.60 mmol) of 2-bromoacetonitrile in 1 mL of THF was added. The reaction was warmed to RT and stirred for additional 1 h. It was quenched by addition of 20 mL of aqueous NH₄Cl and extracted with three 10 mL portions of ethyl acetate. The combined organic extracts were dried over anhydrous sodium sulfate and concentrated. The residue was chromatographed on a silica gel column eluting with ethyl acetate/hexane (1/2) to give compound 32.

### Step 3:

To a stirred solution of 280 mg(1.03 mmol) of compound 32 in 10 mL of oxolane and 5 mL of water were added 21.3 mg (0.10 mmol) of trichlororuthenium and 1.1 g (5.1 mmol) of sodium periodate in portions. The mixture was stirred at RT for 2 h and quenched by addition of 20 mL of water. It was extracted with threel 0 mL portions of ethyl acetate; the combined organic extracts were dried over anhydrous sodium sulfate and concentrated. The residue was chromatographed on a silica gel column eluting with ethyl acetate/petroleum ether (1:4) to give in compound 33.

### Step 4:

To a solution of 50 mg (0.16 mmol) of compound 33 in 3 mL of DMF were added 68 mg (0.49 mmol) K₂CO₃ and 70 mg (0.49 mmol) of Mel. The mixture was stirred at RT overnight and quenched by addition of 10 mL of water. It was extracted with three 10 ml portions of ethyl acetate; the combined organic extracts were dried with Na₂SO₄ and concentrated. The residue was purified by Prep-HPLC (Column, C18 silica gel; mobile phase, ACN/H₂O=60% ; Detector UV 254 nm) to give compound 34.

### Step 5:

To a solution of 400 mg (1.20 mmol) of compound 34 in 4 mL of THF, 4 mL of MeOH and 2 mL of H₂O were added 71 mg (1.32 mmol) of NH₄Cl and 268 mg (4.80 mmol) of iron powder. The mixture was stirred at 60° C. for 3 h. It was filtered and the filtrate was concentrated to give compound 35. LC-MS: m/e=303 [M+H]⁺.

### 9. Synthesis of Intermediate 37:

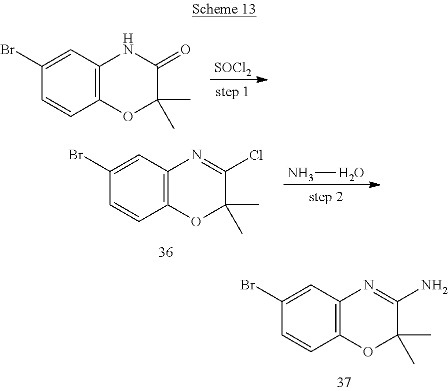

### Steps 1 & 2:

To a stirred solution of 400 mg (1.56 mmol) of 6-bromo-2,2-dimethyl-3,4-dihydro-2H-1,4-benzoxazin-3-one in 14 mL (0.12 mmol) of SOCl₂ was added 0.02 mL of DMF dropwise at RT. The mixture was stirred at 70° C. for 2 h and concentrated under vacuum; the residue was quenched with 10 mL of aqueous ammonium solution. It was extracted with three 30 mL portions of ethyl acetate; the combined organic extracts were washed with brine and dried over anhydrous Na₂SO₄. After filtration, the filtrate was concentrated to afford compound 37. LC-MS: m/e =255 [M+H]⁺.

### 10. Synthesis of Intermediate 42:

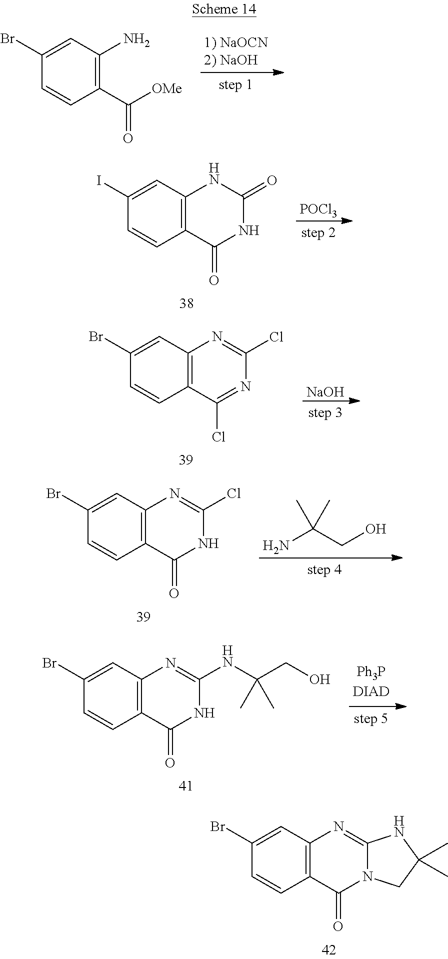

### Step 1:

To a stirred solution of 10.0 g (43.5 mmol) of methyl 2-amino-4-bromobenzoate in 50 mL of acetic acid was added 3.2 g (49.6 mmol) of sodium cyanate in portions. The mixture was stirred at RT for 22 h and diluted with 100 mL of water. The precipitates were collected by filtration and washed with water (3×50 mL). The solid was dissolved in 20 mL of NaOH solution (32%); the mixture was stirred at 100° C. for 4 h and cooled down to RT. The precipitates were collected by filtration and washed with water (3×50 mL) to give compound 38. LC-MS: m/e=241,243 [M+H]⁺.

### Step 2:

A solution of 2.57 g (10.7 mmol) of compound 38 and 0.88 mL (5.3 mmol) of DIPEA in 14.7 mL (157 mmol) of POCl₃ was stirred at 100° C. for 4 h under N₂ atmosphere. The mixture was allowed to cool down to RT and concentrated under vacuum. The residue was quenched with H₂O and extracted with three 50 mL portions of DCM. The combined organic layers were washed with brine and dried over anhydrous Na₂SO₄. It was filtered and the filtrate was concentrated. The residue was purified by silica gel column chromatography eluting with DCM/MeOH (3/2) to afford compound 39. LC-MS: m/e=277, 279 [M+H]⁺.

### Step 3:

A solution of 0.75 g (2.70 mmol) of compound 39 in 7.5 mL of NaOH solution (4% in water) and 2.5 mL of THF was stirred at RT for 2 h. The reaction was quenched with AcOH and extracted with three 20 mL portions of ethyl acetate. The combined organic extracts were concentrated to give compound 40. LC-MS: m/e=259, 261 [M+H]⁺.

### Step 4:

A mixture of 1.0 g (3.85 mmol) of compound 40 in 10 g (112 mmol) of 2-amino-2-methylpropan-1-ol was stirred at 100° C. for 10 h and cooled RT. The mixture was diluted with 50 mL of water and extracted with three 50 mL portions of ethyl acetate. The combined organic extracts were concentrated; the residue was purified by silica gel column chromatography eluting with DCM/MeOH (1/2) to afford compound 41. LC-MS: m/e=312, 314 [M+H]⁺.

### Step 5:

To a stirred mixture of 880 mg (2.82 mmol) of compound 41 and 813 mg (3.10 mmol) of PPh₃ in 20 mL of THF was added 627 mg (3.10 mmol) of DIAD dropwise at 0° C. under N2 atmosphere. The reaction mixture was stirred at RT for 18 h. It was quenched with 20 mL of H₂O and extracted with three 30 mL portions of ethyl acetate. The combined organic extracts were concentrated to give a residue, which was purified by reverse flash chromatography (column, C18 silica gel, mobile phase, A: 0.05% ammonium bicarbonate in Water, B:Acetonitrile, 0% to 10% gradient in 30 mins; detector, UV 254 nm) to afford compound 42. LC-MS: m/e=294, 296 [M+H]⁺.

### LC-MS Conditions Used in the Experimental Procedures Described Above are Listed Below.

Condition A: Shimadzu LC20ADXR/LCMS2020, Column: Kinextex XB-C18 (50*3.0 mm) 2.6 μm; Mobile phase: A: 0.1% Formic acid in Water, B: 0.1% Formic acid in Acetonitrile; Gradient: 90:10 to 0:100 (A:B) over 1.1 min, 0:100 (A:B) for 0.50 min, Flow Rate: 1.5 ml/min. UV detection: 190-400 nm.

Condition B: Shimadzu LC20AD/LCMS2020; Column: Shim-pack XR-ODS (50*3.0 mm) 2.2 μm; Mobile phase: A: 0.05% Trifluoroacetic acid in Water, B: 0.05% Trifluoroacetic acid in Acetonitrile; Gradient: 95:5 to 0:100 (A:B) over 1.1 min, 0:100 (A:B) for 0.55 min, Flow Rate: 1.2 ml/min; UV detection: 190-400 nm.

Condition C: Shimadzu LC30AD/LCMS2020, Column: Ascentis Express (50*3.0 mm) 2.7 μm; Mobile phase: A: 0.05% Trifluoroacetic acid in Water, B: 0.05% Trifluoroacetic acid in Acetonitrile; Gradient: 95:5 to 0:100 (A:B) over 1.2 min, 0:100 (A:B) for 0.50 min, Flow Rate: 1.5 ml/min. UV detection: 190-400 nm.

Condition D: Shimadzu LC20OADXR/LCMS2020, Column: Kinextex XB-C18 (50*3.0 mm) 2.6 μm; Mobile phase: A: 0.1% Formic acid in Water, B: 0.1% Formic acid in Acetonitrile; Gradient: 90:10 to 0:100 (A:B) over 1.1 min, 0:100 (A:B) for 0.50 min, Flow Rate: 1.5 ml/min. UV detection: 190-400 nm.

Condition E: Shimadzu LC20AD/LCMS2020; Column: Shim-pack XR-ODS (50*3.0 mm) 2.2 μm; Mobile phase: A: 0.05% Trifluoroacetic acid in Water, B: 0.05% Trifluoroacetic acid in Acetonitrile; Gradient: 95:5 to 0:100 (A:B) over 1.1 min, 0:100 (A:B) for 0.55 min, Flow Rate: 1.2 ml/min; UV detection: 190-400 nm.

Condition F: Shimadzu LC20ADXR/LCMS2020, Column: Poroshell HPH-C18 (50*3.0 mm) 2.7 μm; Mobile Phase A: 5 mM Ammonium Bicarbonate in Water, Mobile Phase B: Acetonitrile; Gradient: 90:10 to 5:95 (A:B) over 2.1 min, 5:95 (A:B) for 0.60 min; Flow rate: 1.2 mL/min; UV detection: 190-400 nm.

Condition G: LC-MS (Shimadzu LC20ADXR/LCMS2020, Column: Kinextex EVO C18 (50*3.0 mm) 2.6 μm; Mobile phase A: 5 mmol/L Ammonium Bicarbonate in Water, B: Acetonitrile; Gradient: 90:10 to 5:95 (A:B) over 2.0 min, 5:95 (A:B) for 0.60 min, Flow Rate: 1.2m1/min. UV detection: 190-400 nm)

Condition H: LCMS for PH-AGX-104-009-0: (Shimadzu LC2OADXR/LCMS2020, Column: Kinetex EVO C18, 3.0*50 mm, 2.6 μm; Mobile Phase A: 0.04% NH₄OH in water, B: Acetonitrile; Gradient: 90:10 to 5:95 (A:B) over 2.1 min, 5:95 (A:B) for 0.6 min; Flow Rate: 1.2 mL/min; UV detection: 190-400 nm)

Condition I: LC-MS (Shimadzu LC30AD/LCMS2020, Column: CORTECS C18 100A,2.1*50 mm,2.7 μm; Mobile phase A: Water/0.1% FA, Mobile phase B: Acetonitrile/0.1% FA; Flow rate: 1.0 mL/min; Gradient:10% B to 100% B in 2.0 min, hold 0.6 min; 190-400 nm)

### Assays

Protocols that may be used to determine the recited potency for the compounds of the disclosure are describe below.

### PRMT5:MEP50 Flashplate Assay:

Ten-point curves of inhibitor compounds were made using serial threefold dilutions in DMSO (the final top concentration of compound was 10 μM, 1% DMSO). Reaction mixture consisting of 50 mM Tris-HCl (pH 8.5), 0.002% Tween20, 0.005% BSA (Bovine Serum Albumin), 1 mM TCEP, and 1% DMSO. Substrates are freshly prepared in reaction buffer. PRMT5:MEP50 was then added into substrate solution and mixed gently. Inhibitor compound was then added and incubated for 30 min at room temperature. ³H-SAM was added to initiate the reaction. Reactions were incubated for 2 h at room temperature and quenched with 0.5 mM SAM (S-adenosyl-L-Methionine) in assay buffer. An aliquot of the reaction mix was transferred to a streptavidin-coated 384-well FlashPlate (PerkinElmer). After an incubation time of 1 h, the plate was washed and then read on a TopCount (PerkinElmer) to measure the amount of tritium incorporated into the peptide substrate. IC₅₀ was calculated using conventional curve-fitting method. The testing results for selected compounds are summarized in Table 5, wherein A represents the IC₅₀ value of <1.0 nM; B represents the IC₅₀ value of 1.0-100 nM.

TABLE 5
PRMT5:MEP50 Inhibitory Activity of Representative Examples

| | PRMT5 | | PRMT5 |
|---|---|---|---|
| Compound | IC₅₀ | Compound | IC₅₀ |
| JNJ-64619178* | A (0.35 nM) | 1-16 | A |
| 1-7 | A | 1-17 | A |
| 1-8 | A | 1-18 | B |
| 1-9 | A | 2-8 | B |
| 1-10 | A | 2-9 | B |
| 1-11 | A | 2-10 | B |
| 1-12 | A | 2-11 | B |
| 1-13 | A | 2-12 | A |
| 1-14 | A | 3-7 | B |
| 1-15 | B | 4-3 | A |

*JNJ-64619178 is a reference compound, which has CAS# [2086772-26-9].

Unless otherwise indicated, all numbers expressing quantities of ingredients, properties such as molecular weight, reaction conditions, and etc. used in herein are to be understood as being modified in all instances by the term “about.” Each numerical parameter should at least be construed in light of the number of reported significant digits and by applying ordinary rounding techniques. Accordingly, unless indicated to the contrary, the numerical parameters may be modified according to the desired properties sought to be achieved, and should, therefore, be considered as part of the disclosure. At the very least, the examples shown herein are for illustration only, not as an attempt to limit the scope of the disclosure.

The terms “a,” “an,” “the” and similar referents used in the context of describing embodiments of the present disclosure (especially in the context of the following claims) are to be construed to cover both the singular and the plural, unless otherwise indicated herein or clearly contradicted by context. All methods described herein may be performed in any suitable order unless otherwise indicated herein or otherwise clearly contradicted by context. The use of any and all examples, or exemplary language (e.g., “such as”) provided herein is intended merely to better illustrate embodiments of the present disclosure and does not pose a limitation on the scope of any claim. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

Certain embodiments are described herein, including the best mode known to the inventors for carrying out the embodiments. Of course, variations on these described embodiments will become apparent to those of ordinary skill in the art upon reading the foregoing description. The inventor expects skilled artisans to employ such variations as appropriate, and the inventors intend for the embodiments of the present disclosure to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A compound represented by a formula: 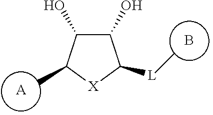
or a pharmaceutically acceptable salt thereof;
wherein 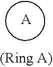
is an optionally substituted 9-membered bicyclic aromatic heterocyclic ring system containing 1, 2, 3, 4, 5, or 6 ring nitrogen atoms; 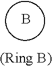
is an optionally substituted fused bicyclic or tricyclic heterocyclic ring system containing 1, 2, 3, 4, 5, or 6 ring heteroatoms independently selected from N, O and S;
X is —O—, —CH₂-, or —CF₂-;
L is optionally substituted C₁₋₃ hydrocarbylene, optionally substituted —O—C₁₋₂ hydrocarbylene-, optionally substituted —S—C₁₋₂ hydrocarbylene-, or optionally substituted —NR^{A}—C₁₋₂ hydrocarbylene-; and
R^{A} is H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl.

2. The compound of claim 1, wherein Ring A comprises: 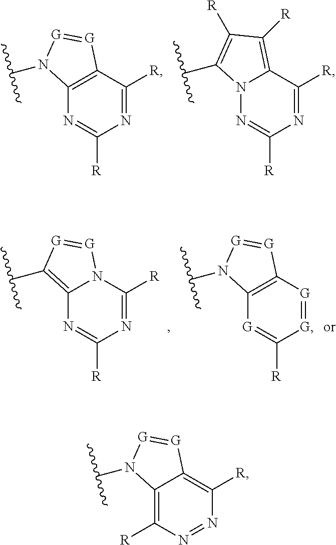
and Ring B comprises: 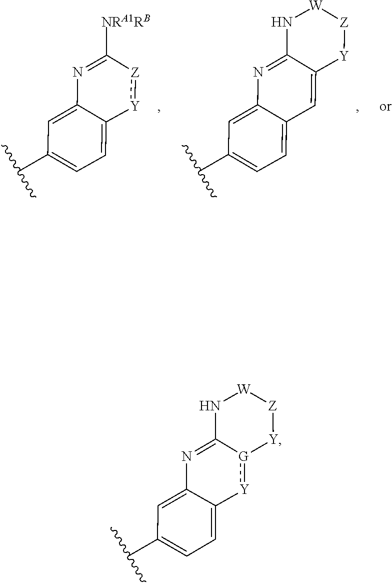
wherein
each structure is optionally substituted;
G is independently N or CR;
Y is independently a bond, —C(R^{C}R^{D})—, CH, —C(═O)—, —O—, —N(R^{A})-, or —S(O)₀₋₂-;
Z is —C(R^{C}R^{D})—, —C(═O)—, C(Br), CH, —O—, —N(R^{A})-, or —S(O)₀₋₂-;
W is —C(R^{C}R^{D})—, —C(═O)—, or —SO₂-;
Dashed line represents optionally with or without a bond;
each R is independently H, F, Cl, Br, I, —NR^{A}R^{B}, C₁₋₆ hydrocarbyl, —OH, —CN, or —O—C₁₋₆ alkyl; each R^{C}, and each R^{D} are independently H, F, Cl, Br, I, —NR^{A}R^{B}, C₁₋₆ hydrocarbyl, —H, —CN, or —O—C₁₋₆ alkyl;
each R^{A} and R^{A1} are independently H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl;
R^{B} is H, C₁₋₆ hydrocarbyl, C₁₋₆ heteroaryl, C₁₋₆ heterocycloalkyl, —C(O)—C₁₋₆ alkyl, —C(O)NH—C₁₋₆ alkyl, or —C(O)OC₁₋₆ alkyl; and
R^{A1} and Z or substituent of Z may connect and together with the ring containing Z to form a fused ring.

3. The compound of claim 1, wherein Ring A comprises optionally substituted 4-amino-7H-pyrrolo[2,3-d]pyrimidin-7-yl.

4. The compound of claim 1, wherein Ring A comprises optionally substituted 6-amino-9H-purin-9-yl.

5. The compound of claim 1, wherein Ring A comprises optionally substituted 7-amino-3H-[1,2,3]triazolo[4,5-d]pyrimidin-3-yl.

6. (canceled)

7. (canceled)

8. The compound of claim 1, wherein Ring B comprises optionally substituted 2-amino-3-methyl-4-oxo-3,4-dihydroquinazolin-7-yl.

9. (canceled)

10. (canceled)

11. (canceled)

12. (canceled)

13. The compound of claim 1, wherein Ring B comprises optionally substituted (S)-3-methyl-3,4-dihydro-2H-[1,4]oxazino[3,2-b]quinolin-7-yl.

14. The compound of claim 1, wherein Ring B comprises optionally substituted (R)-3-methyl-3,4-dihydro-2H-[1,4]oxazino[3,2-b]quinolin-7-yl.

15. The compound of claim 1, wherein Ring B comprises optionally substituted (R)-2-methyl-3,4-dihydro-2H-[1,4]oxazino[3,2-b]quinolin-7-yl.

16. The compound of claim 1, wherein Ring B comprises optionally substituted (S)-2-methyl-3,4-dihydro-2H-[1,4]oxazino[3,2-b]quinolin-7-yl.

17. (canceled)

18. The compound of claim 1, wherein Ring B comprises optionally substituted 3,4-dihydro-2H-[1,4]thiazino[3,2-b]quinolin-7-yl.

19. (canceled)

20. The compound of claim 1, wherein Ring B comprises optionally substituted 2-amino-3-bromoquinolin-7-yl.

21. The compound of claim 1, wherein Ring B comprises optionally substituted 2-amino-3-cyclopropyl-4-oxo-3,4-dihydroquinazolin-7-yl.

22. (canceled)

23. The compound of claim 1, wherein X is —CH₂-.

24. The compound of claim 1, wherein X is —O-.

25. (canceled)

26. The compound of claim 1, wherein L is —CH₂—CH₂-.

27. The compound of claim 1, wherein L is —CH₂—CH₂—CH₂-

28. The compound of claim 1, wherein L is —CH₂O-, or —O—CH₂-.

29. (canceled)

30. The compound of claim 1, or a pharmaceutically acceptable salt thereof, wherein the compound is optionally substituted 7-(2-((1S,2R,3S,4R)-2,3-dihydroxy-4-(7H-pyrrolo[2,3-d]pyrimidin-7-yl)cyclopentyl)ethyl)quinazolin- 4(3H)-one, optionally substituted (1S,2R,3S,5R)-3-(2-(3,4-dihydro-2H-[1,4]oxazino[3,2-b]quinolin-7-yl)ethyl)-5-(7H-pyrrolo[2,3-d]pyrimidin-7-yl)cyclopentane-1,2-diol, optionally substituted 7-(2-((1S,2R,3S,4R)-2,3-dihydroxy-4-(7H-pyrrolo[2,3-d]pyrimidin-7-yl)cyclopentyl)ethyl)-2H-[1,4]oxazino[3,2-b]quinolin-3(4H)-one, optionally substituted (1S,2R,3S,5R)-3-(2-(3,4-dihydro-2H-[1,4]thiazino[3,2-b]quinolin-7-yl)ethyl)-5-(7H-pyrrolo[2,3-d]pyrimidin-7-yl)cyclopentane-1,2-diol, optionally substituted 6-(2-((1S,2R,3S,4R)-2,3-dihydroxy-4-(7H-pyrrolo[2,3-d]pyrimidin-7-yl)cyclopentyl)ethyl)-2H- benzo[e][1,2,4]thiadiazine 1,1-dioxide, optionally substituted (1R,2S,3R,5S)-3-(7H-pyrrolo[2,3-d]pyrimidin-7-yl)-5-(3-(quinolin-7- yl)propyl)cyclopentane-1,2-diol, optionally substituted 7-(2-((1S,2R,3S,4R)-2,3-dihydroxy-4-(7H-pyrrolo[2,3-d]pyrimidin-7-yl)cyclopentyl)ethyl)-2H- [1,4]thiazino[3,2-b]quinolin-3(4H)-one, optionally substituted 6-(2-((2R,3S,4R,5R)-3,4-dihydroxy-5-(7H-pyrrolo[2,3-d]pyrimidin-7-yl)tetrahydrofuran-2-yl)ethyl)-2H-benzo[e][1,2,4]thiadiazine 1,1-dioxide, optionally substituted 8-(2-((1S,2R,3S,4R)-2,3-dihydroxy-4-(7H-pyrrolo[2,3-d]pyrimidin-7-yl)cyclopentyl)ethyl)-2,3-dihydroimidazo[2,1-b]quinazolin-5(1H)-one, optionally substituted (2R,3S,4R,5R)-2-(2-(2H-benzo[b][1,4]oxazin-6-yl)ethyl)-5-(7H-pyrrolo[2,3-d]pyrimidin-7-yl)tetrahydrofuran-3,4- diol, or optionally substituted 6-(2-((1S,2R,3S,4R)-2,3-dihydroxy-4-(7H-pyrrolo[2,3-d]pyrimidin-7-yl)cyclopentyl)ethyl)-2H-benzo[b][1,4]thiazine 1,1-dioxide.

31. (canceled)

32. (canceled)

33. The compound of claim 1, wherein the compound is deuterated.

34. (canceled)

35. (canceled)

36. (canceled)

37. (canceled)

38. (canceled)

39. (canceled)

40. (canceled)

41. (canceled)

42. A compound, or a pharmaceutically acceptable salt thereof, wherein the compound is:

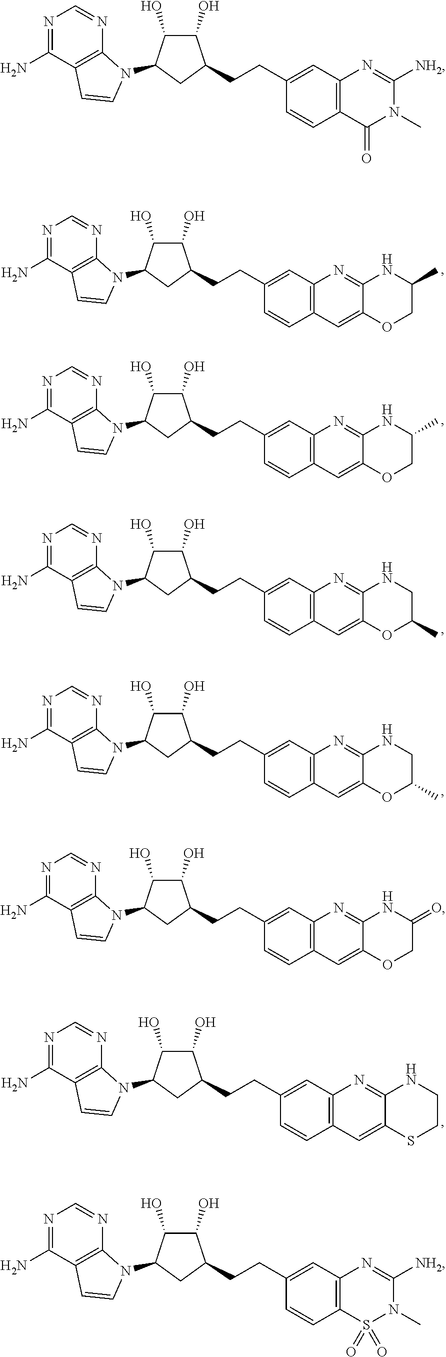

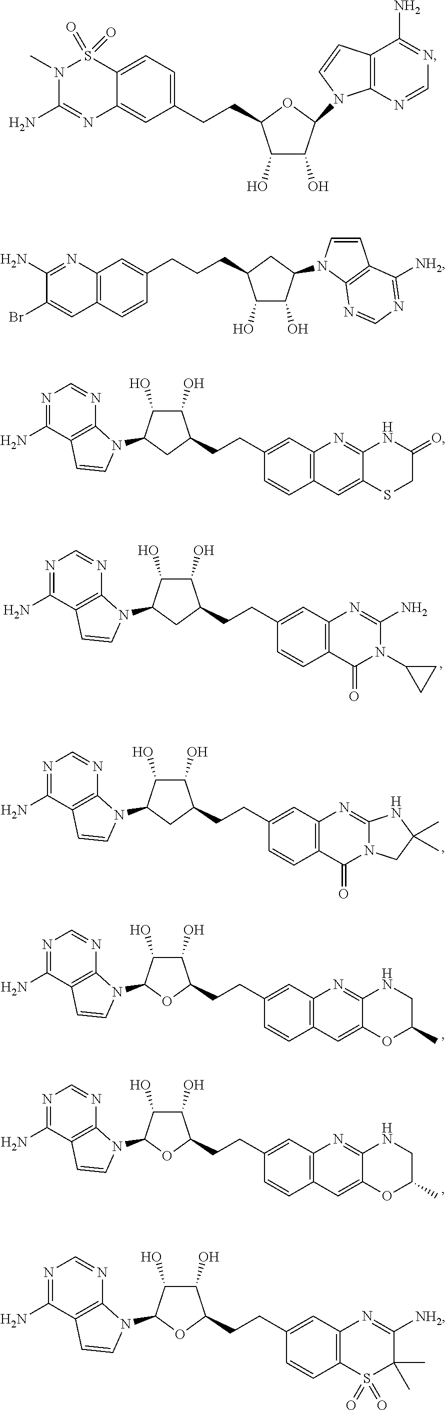

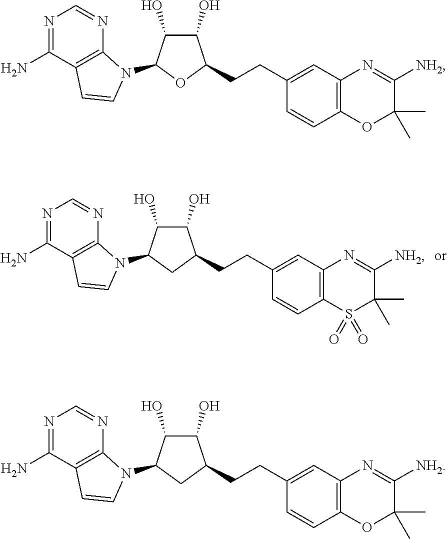 [SMILES: CC1(C)Oc2ccc(CC[C@H]3O[C@@H](n4ccc5c(N)ncnc54)[C@H](O)[C@@H]3O)cc2N=C1N ; CC1(C)C(N)=Nc2cc(CC[C@H]3C[C@@H](n4ccc5c(N)ncnc54)[C@H](O)[C@@H]3O)ccc2S1(=O)=O ; CC1(C)Oc2ccc(CC[C@H]3C[C@@H](n4ccc5c(N)ncnc54)[C@H](O)[C@@H]3O)cc2N=C1N]

43. A method of treating cancer, an infectious disease, another PRMT5-related disease or disorder, or a combination thereof, comprising administering a compound of claim 1, to a mammal in need thereof.

44. (canceled)

45. (canceled)

46. (canceled)

47. (canceled)
